SEQUENCE LISTING (6 sequences)

SEQ ID NO: 1 - protein, 430 aa, Saccharomyces cerevisiae
MAEKILENKDPKLEAMTVDHEVSAPKPIPVDEPTLTRVARPLRHVRHIPVKSLVFHSKHGPITFSYENKIKLPISKNKLVVQVNYVGLNPVDMKIRNGYTKPIYGEAGIGREYSGVITHVGDNLTNRWNVGDDVYGIYYHPKLAIGALQSSLLIDPRVDPILMRPKNTLSPEKAAGSLFCLGTALNLLAQLKEKDQLNTESNVLINGGTSSVGMFAIQLLKRYYKVSKKLVVVTSGNGAAVLSEHFPDLKDEIIFINYLSCRGKSSKPLRRMLDTGKVVDYDDFNTLKETEDYTQGKFNVVLDFIGGYDILSHSSSLIHAKGAYITTVGDYVGNYKKDVFDSWDNPSANARKMFGSMLWSYDYSHFYFDPNIKIIPKKNDWIHECGKLLNEGVVDCVVDKVYSWKNFKEAFSYMATQRAQGKLIMKVEGF

SEQ ID NO: 2 - protein, 430 aa, Saccharomyces cerevisiae
MAEKILENKDPKLEAMTVDHEVSAPKPIPVDEPTLTRVARPLRHVRHIPVKSLVFHSKHGPITFSYENKIKLPISKNKLVVQVNYVGLNPVDMKIRNGYTKPIYGEAGIGREYSGVITHVGDNLTNRWNVGDDVYGIYYHPKLAIGALQSSLLIDPRVDPILMRPKNTLSPEKAAGSLFCLGTALNLLAQLKEKDQLNTESNVLINGGTSSVGMFAIQLLKRYYKVSKKLVVVTSGNGAAVLSEHFPDLKDEIIFINYLSCRGKSSKPLRRMLDTGKVVDYDDFNTLKETEDYTQGKFNVVLDFIGGYDILSHSSSLIHAKGAYITTVGDYVGNYKKDVFDSWDNPSANARKMFGSMLWSYDYSHFYFDPNIKIIPKKNDWIHECGKLLNEGVVDCVVDKVYSWKIFKEAFSYMATQRAQGKLIMKVEGF

SEQ ID NO: 3 - protein, 429 aa, Saccharomyces cerevisiae
MAKDILKNQDPKLQAMIVEHSAPAPKEIPMDAPVLKRVARPLRHVKFIPIKSLIFHTKTGPMDFSYEKKIKTPIPKNKIVVRVSNVGLNPVDMKIRNGYTSSIYGEIGLGREYSGVITEVGENLNYAWHVGDEVYGIYYHPHLAVGCLQSSILVDPKVDPILLRPESVSAEEAAGSLFCLATGYNILNKLSKNKYLKQDSNVLINGGTSSVGMFVIQLLKRHYKLQKKLVIVTSANGPQVLQEKFPDLADEMIFIDYLTCRGKSSKPLRKMLEEKKISQYDPVEDKETILNYNEGKFDVVLDFVGGYDILSHSSSLIHGGGAYVTTVGDYVANYKEDIFDSWDNPSANARKMFGSIIWSYNYTHYYFDPNAKTASANNDWIEQCGDFLKNGTVKCVVDKVYDWKDHKEAFSYMATQRAQGKLIMNVEKF

SEQ ID NO: 4 - protein, 429 aa, Saccharomyces cerevisiae
MAKDILKNQDPKLQAMIVEHSAPAPKEIPMDAPVLKRVARPLRHVKFIPIKSLIFHTKTGPMDFSYEKKIKTPIPKNKIVVRVSNVGLNPVDMKIRNGYTSSIYGEIGLGREYSGVITEVGENLNYAWHVGDEVYGIYYHPHLAVGCLQSSILVDPKVDPILLRPESVSAEEAAGSLFCLATGYNILNKLSKNKYLKQDSNVLINGGTSSVGMFVIQLLKRHYKLQKKLVIVTSANGPQVLQEKFPDLADEMIFIDYLTCRGKSSKPLRKMLEEKKISQYDPVEDKETILNYNEGKFDVVLDFVGGYDILSHSSSLIHGGGAYVTTVGDYVANYKEDIFDSWDNPSANARKMFGSIIWSYNYTHYYFDPNAKTASANNDWIEQCGDFLKNGTVKCVVDKVYDWKIHKEAFSYMATQRAQGKLIMNVEKF

SEQ ID NO: 5 - DNA, 20 bp, Artificial Sequence
  gRNA
ttattcctggaaaaatttca

SEQ ID NO: 6 - DNA, 20 bp, Artificial Sequence
  gRNA
tataagaaaatgcttcttta

(19) United States
(12) Patent Application Publication
(10) Pub. No.: US 2023/0227769 A1
(43) Pub. Date: Jul. 20, 2023

(54) Means and Methods to Improve Yeast Fermentation Efficiency

(71) Applicants: VIB VZW, Gent (BE); KATHOLIEKE UNIVERSITEIT LEUVEN, K.U.LEUVEN R&D, Leuven (BE)

(72) Inventors: Johan Thevelein, Blanden (BE); Maria Remedios Foulquié Moreno, Brussel (BE); Gert Vanmarcke, Leuven (BE); Quinten Deparis, Heverlee (BE)

(21) Appl. No.: 18/010,677

(22) PCT Filed: Jun. 15, 2021
(86) PCT No.: PCT/EP2021/066118

§ 371 Date: Dec. 15, 2022

(30) Foreign Application Priority Data
Jun. 15, 2020 (EP) 20179988.9

(51) Int. Cl. C12N 1/16; C12N 15/81; C12N 1/22; C12P 7/10

(52) U.S. Cl. CPC C12N 1/16; C12N 15/81; C12N 1/22; C12P 7/10

(57) ABSTRACT

The invention relates to the field of microbiology, more particularly to fermentation technology. Yeast fermentation, particularly production of bio-based compounds starting from second generation carbon sources is often hampered by the presence of inhibitory chemicals. This application provides means and methods to overcome the negative effect of fermentation inhibitors, more particularly by providing chimeric genes and yeast strains comprising them that are tolerant to these inhibitors.

### CROSS-REFERENCE TO RELATED APPLICATIONS

This application is a national phase entry under 35 U.S.C. § 371 of International Patent Application PCT/EP2021/066118, filed Jun. 15, 2021, designating the United States of America and published in English as International Patent Publication WO 2021/255029 on Dec. 23, 2021, which claims the benefit under Article 8 of the Patent Cooperation Treaty to European Patent Application Serial No. 20179988.9, filed Jun. 15, 2020, the entireties of which are hereby incorporated by reference.

### FIELD OF THE INVENTION

The invention relates to the field of microbiology, more particularly to fermentation technology. Yeast fermentation, particularly production of bio-based compounds starting from second generation carbon sources is often hampered by the presence of inhibitory chemicals. This application provides means and methods to overcome the negative effect of fermentation inhibitors, more particularly by providing chimeric genes and yeast strains comprising them that are tolerant to these inhibitors.

### BACKGROUND

Renewable biomass, including lignocellulosic material and agricultural residues such as corn fiber, corn stover, corn cob, wheat straw, rice straw, and sugarcane bagasse, are low cost materials for a biobased economy. Second-generation bioethanol for the transport sector and bio-based compounds replacing petroleum-based plastics are promising alternative products with multiple major benefits over fossil fuels and first-generation bioethanol. However, for cost-efficient production of second-generation (2G) bioethanol several hurdles have to be overcome. One of those is the high level of inhibitors present in lignocellulose hydrolysates that severely reduce the yeast fermentation rate and yield, in particular that of xylose (Bellissimi et al. 2009 FEMS Yeast Res 9: 358-364). These inhibitors, which included acetic acid, hydroxymethylfurfural (HMF), furfural, formic acid, levulinic acid, vanillin, 4-hydroxybenzaldehyde and 4-hydroxybenzoic acid are produced during the pretreatment of the lignocellulosic biomass. The aldehyde group in HMF and furfural for example affects DNA, RNA, proteins and membranes, and causes accumulation of reactive oxygen species (Allen et al. 2010 Biotechnol Biofuels 3: 2; Janzowski et al. 2000 Food Chem Toxicol 38: 801-809). Moreover, HMF inhibits activity of multiple enzymes, negatively affects lag phase length and induces apoptosis (Modig et al. 2002 Biochem J 363: 769-776.). High temperatures which are preferred to maintain adequate activity of lignocellulolytic enzymes during the simultaneous saccharification, fermentation and consolidated bioprocessing, further increase the toxicity of the inhibitory compounds. As the chemicals aggravate the burden for the yeast, it would be advantageous to develop new yeast strains with an innate tolerance for fermentation inhibitors.

Several alleles and mechanisms have been disclosed that could provide some level of tolerance against furfural and/or HMF (WO200511214A1, WO2009006135A2, WO2012135420A2). Also for acetic acid, detoxification processes have been studied (Pampulha & Loureiro-Dias 1989 Appl Microbiol Biotechnol 31: 547-550) and alleles identified that provide tolerance (WO2016083397). However there is still a need for additional tolerance alleles and especially a need for yeast strains that are tolerant to multiple fermentation inhibitors.

### SUMMARY

Here, the Applicants report on mutant AST alleles that when expressed in yeast confer tolerance to HMF and furfural but also to other inhibitors present in lignocellulose hydrolysates, like formic acid, vanillin and acetic acid. Hence, any cell factory yeast strain developed for the production of a bio-based chemical starting from lignocellulosic biomass, will profit from the presence of the mutant AST2 (and optionally the additional presence of a mutant AST1 allele) herein disclosed because of the increased inhibitor tolerance provided.

However, the findings herein disclosed have also high application potential for the improvement of industrial yeast strains for first generation production of bioethanol and bio-based chemicals. First generation (1G) bioethanol production for example starting from molasses can get compromised because of high concentrations of acetic acid produced by contaminating bacteria accumulating due to water recycling practices. Expression of the AST2^{N406I} mutant allele improves acetic acid tolerance (see Example 7) and thus overcomes this problem. An additional advantage of the AST2^{N406I} mutation for 1G bioethanol production (e.g. starting from corn mash) is that its expression enhances the ethanol titer while reducing yeast biomass production. Moreover, the application discloses that AST2^{N406I} expression reduces the production of acetaldehyde in wort fermentations. Acetaldehyde is an unwanted compound in beer. Hence, the herein disclosed findings can be used to improve brewer's yeast strains.

In a first aspect the application provides the Ast^{N406I} protein as depicted in SEQ ID No. 2 as well as the nucleic acid molecule encoding SEQ ID No. 2. Also a chimeric gene is provided comprising a promoter which is active in a eukaryotic cell, a nucleic acid molecule encoding an amino acid sequence with a sequence identity of at least 90% to SEQ ID No. 1, said amino acid sequence comprises an isoleucine residue on position 406 of SEQ ID No. 1, and a 3′ end region involved in transcription termination or polyadenylation. In a particular embodiment, the nucleic acid molecule of said chimeric gene encodes SEQ ID No. 2. Also a vector comprising the nucleic acid molecule or the chimeric gene is provided.

In another aspect the application provides improved yeast strains. In one embodiment, said improved yeast strains comprise the above nucleic acid molecules, chimeric genes or vectors. In another embodiment, the application provides a xylose fermenting yeast comprising an amino acid sequence with a sequence identity of at least 90% to SEQ ID No. 1, said amino acid sequence comprises an isoleucine residue on position 406 of SEQ ID No. 1, more particularly comprises SEQ ID No. 2. In another embodiment, said yeasts are provided for metabolizing lignocellulosic hydrolysates comprising one or more growth inhibiting compounds selected from the list consisting of HMF, furfural, formic acid, acetic acid, levulinic acid, 4-hydroxybenzoic acid, 4-hydroxybenzaldehyde and vanillin. The application also provides biologically pure cultures of the yeasts and a culture comprising lignocellulosic hydrolysates and any of the above described yeast strains. It is further disclosed herein that the tolerance of the above described yeasts towards one or more fermentation inhibitors can be further improved by the additional expression of a mutant AST1 allele. Hence the above yeasts are provided further comprising a nucleic acid molecule encoding an amino acid sequence with a sequence identity of at least 90% to SEQ ID No. 3, said amino acid sequence comprises an isoleucine residue on position 405 of SEQ ID No. 3, or more particularly comprises a nucleic acid molecule encoding SEQ ID No. 4.

In yet another aspect the use is provided of a nucleic acid molecule encoding an Ast2 protein comprising an N406I mutation to provide in yeast tolerance to a fermentation inhibitor selected from the list consisting of HMF, furfural, formic acid, acetic acid, levulinic acid, 4-hydroxybenzoic acid, 4-hydroxybenzaldehyde and vanillin. Uses of the disclosed yeast strains are also provided to produce a fermentation product from lignocellulosic hydrolysates. In line with the above, a method is provided of producing a fermentation product, the method comprises the step of fermenting a medium comprising a carbon source and one or more growth inhibiting compounds selected from the group consisting of HMF, furfural, formic acid, acetic acid, levulinic acid, 4-hydroxybenzoic acid, 4-hydroxybenzaldehyde and vanillin, wherein any of the yeasts herein disclosed ferments or metabolizes the carbon source to said fermentation products; and optionally the step of recovering the fermentation product. In one embodiment, the fermentation product referred to herein can be ethanol, isobutanol, lactic acid, 2,3-butanediol, muconic acid, protocatechuic acid, 3-hydroxy-propionic acid, acrylic acid, acetic acid, succinic acid, citric acid, amino acids, 1,3-propane-diol, ethylene, glycerol, butyric acid, caproate, butanol, glyoxylate, fatty alcohols, fatty acids, β-lactam antibiotics or cephalosporins. Also the fermentation products produced by said methods are provided.

In yet another aspect, a method is provided to produce a yeast strain able to tolerate the presence of one or more growth inhibiting compounds selected from the list consisting of HMF, furfural, formic acid and acetic acid, the method comprising the step of expressing at least one nucleic acid molecule encoding an amino acid sequence with a sequence identity of at least 90% to SEQ ID No. 1, said amino acid sequence comprises an isoleucine residue on position 406 of SEQ ID No. 1, in said yeast.

The application also provides a mutant AST1 allele, more particularly a nucleic acid molecule encoding an amino acid sequence with a sequence identity of at least 90% to SEQ ID No. 3, said amino acid sequence comprises an isoleucine residue on position 405 of SEQ ID No. 3. The application also provides a mutant Ast1^{D405I} protein and a nucleic acid molecule encoding SEQ ID No. 4. It is disclosed herein that the expression of said AST1 allele further improves tolerance in yeasts expressing one of the herein disclosed AST2 mutant alleles towards one or more inhibitors from the list consisting of HMF, furfural, formic acid, acetic acid, levulinic acid, 4-hydroxybenzoic acid, 4-hydroxybenzaldehyde and vanillin. In a final aspect chimeric genes and yeasts comprising any of the herein disclosed mutant AST1 alleles are provided.

### BRIEF DESCRIPTION OF THE FIGURES

FIGS. 1A-1H shows the fermentation performance (10 ml fermentations, pH 5.2, 35° C., 350 rpm and initial OD 5.0) of 2G yeast strains MD4 (FIGS. 1A-1D) and T18 (FIGS. 1E-1H) in corn cob hydrolysate. The medium was spiked with different industrially relevant concentrations of (FIG. 1A, FIG. 1E) HMF, (FIG. 1B, FIG. 1F) furfural, (FIG. 1C, FIG. 1G) formic acid and (FIG. 1D, FIG. 1H) acetic acid.

FIGS. 2A and 2B illustrates the fermentation performance of the yeast strains with highest HMF tolerance and control strains in small-scale semi-anaerobic fermentations (10 ml, pH 5.2, 35° C. and initial OD 5.0) in the presence of a high HMF concentration (YPD6.5% with 8 g/l HMF). FIG. 2A shows the performance of a selection of S. cerevisiae strains while FIG. 2B shows that of non-conventional yeast species incl. C. glabrata JT26560 and the S. cerevisiae MD4 control strain.

FIGS. 3A and 3B shows the fermentation performance (small-scale fermentations (10 ml), pH 5.2, 35° C., 350 rpm and initial OD 5.0) of whole genome (WG) transformant GVM0, donor C. glabrata JT26560 and recipient MD4 in corn cob hydrolysate enriched with 1 g/l HMF (FIG. 3B) or in the absence of spiked HMF (FIG. 3A).

FIG. 4 shows the fermentation performance (small-scale fermentations (10 ml), pH 5.2, 35° C., 350 rpm and initial OD 5.0) in YPDX medium enriched with 12.0 g/l HMF of WG transformant GVM1 and the two hemizygous GVM1 strains with either the mutant AST2^{N406I} allele or the wild-type AST2 allele.

FIGS. 5A-5E shows the fermentation performance (small-scale fermentations (10 ml), pH 5.2, 35° C., 350 rpm and initial OD 5.0) in YPDX medium enriched with 4.0 g/l furfural (FIG. 5A) or 4.5 g/l vanillin (FIG. 5B), and in corn cob hydrolysate enriched with 0.0 g/l HMF (FIG. 5C), 1.0 g/l HMF (FIG. 5D), or 1.0 g/l furfural (FIG. 5E) of the two hemizygous strains of WG transformant GVM1 containing either the mutant AST2^{N406I} allele or the wild-type AST2 allele.

FIGS. 6A-6D shows the fermentation performance (small-scale fermentations (10 mL), pH 4.76, 35° C., 350 rpm, initial OD600 of 5.0) of JT 28541 (AST2^{wild type}/AST2^{wild type}) and JT 29040 (JT 28541 AST2^{N406I}/AST2^{N406I}) in molasses medium (35% sugarcane molasses) additionally spiked with 1.5 g/l acetic acid (FIG. 6A) or with 2.0 g/l acetic acid (FIG. 6B).

FIGS. 7A-7C shows the fermentation performance (small-scale fermentations (10 ml), 35° C., 350 rpm and initial OD 5.0) of MD4, of MD4 with one AST2^{N406I} allele (MD4.1), of MD4 with four copies of AST2^{N406I} (MD4.4) and of the GVM0 WG transformant in the presence of HMF (7A, YPDX medium enriched with 12.0 g/l HMF at pH 5.2), furfural (FIG. 7B, YPDX with 4.0 g/l furfural at pH 5.2) or a mixture of inhibitors (FIG. 7C, YPDX enriched with a mixture of 2.80 g/l HMF, 1.75 g/l furfural, 0.35 g/l vanillin and 4.20 g/l acetic acid at pH 4.6).

FIGS. 8A-8C shows the fermentation performance (small-scale fermentations (10 ml), 35° C., 350 rpm and initial OD 5.0) of the industrial 2G industrial yeast strain TMB 3400 comprising the mutant AST2^{N406I} allele. FIG. 8A shows the fermentation performance in YPDX medium enriched with 12.0 g/l HMF at pH 5.2, FIG. 8B that in YPDX with 4.0 g/l furfural at pH 5.2, and FIG. 8C in YPDX enriched with a mixture of 2.80 g/l HMF, 1.75 g/l furfural, 0.35 g/l vanillin and 4.20 g/l acetic acid at pH 4.6.

FIG. 9 shows the fermentation performance (small-scale fermentations (10 mL), pH 4.6, 35° C., 350 rpm, initial OD600 of 5.0) of DE-4 AST2^{wild-type}/AST2^{wild-type} and DE-4 AST2^{N406I}/AST2^{N406I} in YPDX with a mixture of 2.80 g/l HMF, 1.75 g/l furfural, 0.35 g/l vanillin and 4.20 g/l acetic acid.

FIGS. 10A-10D shows the fermentation performance of MD4 and MD4.4 in wort. Static fermentations (in flasks with a water lock placed on top) were performed in 250 ml wort (i.e. 70% BME, 30% HMS, 7.2 μM ZnSO₄, 20 ppm O₂) at 14° C. and 120 rpm. GC analysis was performed of (FIG. 10A) acetaldehyde concentrations and (FIG. 10B) ethanol production. Absorbance at wavelength 600 nm was measured to determine biomass formation as depicted in panel (FIG. 10C). Maltose utilization (FIG. 10D) was measured by HPLC.

FIG. 11 shows the fermentation performance (small-scale fermentations (10 mL), pH 5.2, 35° C., 350 rpm and initial OD 5.0) of GVM1 and eight S. cerevisiae strains also containing the AST2^{N406I} mutation in YPDX medium enriched with 12.0 g/l HMF.

FIGS. 12A and 12B shows the fermentation performance (small-scale fermentations (10 ml), pH 4.6, 35° C., 350 rpm and initial OD 5.0) of MD4, GVM1, MD4 comprising 4 AST1^{D405I} copies and GVM1 comprising two AST1^{D405I} copies in YPDX in the presence of an inhibitor cocktail at (FIG. 12A) a low (2.80 g/l HMF, 1.75 g/l furfural, 0.35 g/l vanillin and 4.20 g/l acetic acid) or (FIG. 12B) a high (3.36 g/l HMF, 2.10 g/l furfural, 0.42 g/l vanillin and 5.04 g/l acetic acid) concentration.

### DETAILED DESCRIPTION

### Definitions

The present invention will be described with respect to particular embodiments and with reference to certain drawings but the invention is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

The following terms or definitions are provided solely to aid in the understanding of the invention. Unless specifically defined herein, all terms used herein have the same meaning as they would to one skilled in the art of the present invention. Practitioners are particularly directed to Michael R. Green and Joseph Sambrook, Molecular Cloning: A Laboratory Manual, 4th ed., Cold Spring Harbor Laboratory Press, Plainsview, N.Y. (2012); and Ausubel et al., Current Protocols in Molecular Biology (Supplement 47), John Wiley & Sons, New York (1999), for definitions and terms of the art. The definitions provided herein should not be construed to have a scope less than understood by a person of ordinary skill in the art.

When “sequence identity” of two related nucleotide or amino acid sequences expressed as a percentage is used herein, it refers to the number of positions in the two optimally aligned sequences which have identical residues (×100) divided by the number of positions compared. A gap, i.e. a position in an alignment where a residue is present in one sequence but not in the other is regarded as a position with non-identical residues. The alignment of the two sequences is performed by the Needleman and Wunsch algorithm (Needleman and Wunsch 1970 J Mol Biol 48: 443-453). The computer-assisted sequence alignment above, can be conveniently performed using standard software program such as GAP which is part of the Wisconsin Package Version 10.1 (Genetics Computer Group, Madison, Wis., USA) using the default scoring matrix with a gap creation penalty of 50 and a gap extension penalty of 3. Sequences that have an identity of 100% are identical.

A “promoter” comprises regulatory elements, which mediate the expression of a nucleic acid molecule. For expression, the nucleic acid molecule must be linked operably to or comprise a suitable promoter which expresses the gene at the right point in time and with the required spatial expression pattern. The term “operably linked” as used herein refers to a functional linkage between the promoter sequence and the gene of interest (e.g. the nucleic acid sequence encoding Ast2^{N406I}) such that the promoter sequence is able to initiate transcription of the gene of interest. A promoter that enables the initiation of gene transcription in a eukaryotic cell is referred to as being “active”. To identify a promoter which is active in a eukaryotic cell, the promoter can be operably linked to a reporter gene after which the expression level and pattern of the reporter gene can be assayed. Suitable well-known reporter genes include for example beta-glucuronidase, beta-galactosidase or any fluorescent protein. The promoter activity is assayed by measuring the enzymatic activity of the beta-glucuronidase or beta-galactosidase. Alternatively, promoter strength may also be assayed by quantifying mRNA levels or by comparing mRNA levels of the nucleic acid, with mRNA levels of housekeeping genes such as 18S rRNA, using methods known in the art, such as Northern blotting with densitometric analysis of autoradiograms, quantitative real-time PCR or RT-PCR (Heid et al. 1996 Genome Methods 6: 986-994).

As used herein, “nucleic acid” includes reference to a deoxyribonucleotide or ribonucleotide polymer in either single- or double-stranded form, and unless otherwise limited, encompasses known analogues having the essential nature of natural nucleotides in that they hybridize to single-stranded nucleic acids in a manner similar to naturally occurring nucleotides (e.g. peptide nucleic acids).

By “encoding” or “encodes” or “encoded”, with respect to a specified nucleic acid, is meant comprising the information for transcription into an RNA and in some embodiments, translation into the specified protein or amino acid sequence. A nucleic acid encoding a protein may comprise non-translated sequences (e.g. introns) within translated regions of the nucleic acid, or may lack such intervening non-translated sequences (e.g. as in cDNA). The information by which a protein is encoded is specified by the use of codons. Typically, the amino acid sequence is encoded by the nucleic acid using the “universal” genetic code.

The term “a 3′ end region involved in transcription termination or polyadenylation” encompasses a control sequence which is a DNA sequence at the end of a transcriptional unit which signals 3′ processing or polyadenylation of a primary transcript and is involved in termination of transcription. The control sequence for transcription termination or terminator can be derived from a natural gene or from a variety of genes. For expression in yeast the terminator to be added may be derived from, for example, the TEF or CYC1 genes or alternatively from another yeast gene or less preferably from any other eukaryotic or viral gene.

“Second-generation substrates” as used herein are lignocellulosic biomass or woody crops, agricultural residues, non-foodstuffs or waste, especially lignocellulosic waste streams. Lignocellulosic refers to plant biomass composed of carbohydrate polymers (cellulose, hemicellulose) and an aromatic polymer (lignin). These carbohydrate polymers contain different sugar monomers (six and five carbon sugars) and they are tightly bound to lignin. Lignocellulosic biomass can be broadly classified into virgin biomass, waste biomass and energy crops. Virgin biomass includes all naturally occurring terrestrial plants such as trees, bushes and grass. Waste biomass is produced as a low value by-product of various industrial sectors such as agricultural (corn stover, sugarcane bagasse, straw etc.), forestry (sawmill and paper mill discards). Energy crops are crops with high yield of lignocellulosic biomass produced to serve as a raw material for production of second-generation biofuel, non limiting examples are poplar trees, willow trees, switch grass (Panicum virgatum) and Elephant grass. “Second-generation biofuels” are biofuels produced from second-generation substrates. Fermentation of second-generation substrates can be convincingly evaluated by analysis of the substrate content and metabolites by high performance liquid chromatography (HPLC) as described in the materials and methods section of the present application. Fermentation is then defined as a process during which the level of one or more substrate components (e.g. glucose, xylose) is decreased and the level of one or more metabolites (e.g. ethanol, glycerol) is increased.

The terms “increase”, “obtain”, “improve” or “enhance” herein used are interchangeable and shall mean, in the sense of increasing tolerance in a yeast cell towards one or more fermentation inhibitors described herein or in the sense of increasing the production of a fermentation product, that the yeast comprising the AST2^{N406I} and/or AST1^{D405I} allele has a statistically significantly (p<0.5) or at least 3%, 4%, 5%, 6%, 7%, 8%, 9%, 10%, 15%, 20%, 25%, 30%, 40%, 50%, 60%, 70%, 80%, 90%, or 100% higher yield and/or growth or higher production of the fermentation product compared to control yeast cells at the same growing or fermentation conditions. The skilled person is familiar by identifying control yeast cells which in this case would be genetical identical except for the presence of the AST2^{N406I} and/or AST1^{D405I} allele. The terms “decrease”, “decreased”, “reduce”, “reduction” or “reducing” are interchangeable and shall mean, in the sense of reducing the production of acetaldehyde described herein, that the yeast comprising the AST2^{N406I} has a statistically significantly (p<0.5) or at least 3%, 4%, 5%, 6%, 7%, 8%, 9%, 10%, 15%, 20%, 25%, 30%, 40%, 50%, 60%, 70%, 80%, 90%, or 100% lower production of acetaldehyde compared to control yeast cells at the same growing or fermentation conditions. The skilled person is familiar by identifying control yeast cells which in this case would be genetical identical except for the AST2^{N406I} allele.

The term “statistically significant” or “statistically significantly” different is well known by the person skilled in the art. Statistical significance plays a pivotal role in statistical hypothesis testing. It is used to determine whether the null hypothesis should be rejected or retained. It states that the results are obtained because of chance and are not supporting a real change or difference between two data sets. The null hypothesis is the default assumption that what one is trying to prove did not happen. In contrast the alternative hypotheses states that the obtained results support the theory being investigated. For the null hypothesis to be rejected (and thus the alternative hypothesis to be accepted), an observed result has to be statistically significant, i.e. the observed p-value is less than the pre-specified significance level α. The p stands for probability and measures how likely it is that the null hypothesis is incorrectly rejected and thus that any observed difference between data sets is purely due to chance. In most cases the significance level α is set at 0.05.

### Chimeric Genes Comprising AST2^{N406I}

A major hurdle for economically viable 2G bioethanol production is the presence of high levels of inhibitors in lignocellulose hydrolysates. The inventors of current application identified HMF, furfural, formic and acetic acid as strongest fermentation inhibitors present in 2G substrates. A prolonged lag phase was observed, and the total fermentation yield was significantly reduced (Example 1). To provide a solution the inventors of current invention screened more than 2500 yeast strains for growth in the presence of 8 g/l HMF. Only 15 strains were able to withstand such high HMF concentrations. Interestingly, the majority of these strains were also furfural tolerant. This is most likely due to the similar toxicity that both furan aldehydes exert. Their detoxification mechanisms, including the action of aldehyde reductases, also show considerable overlap between HMF and furfural. Genomic DNA from the most tolerant strains was used in a whole genome transformation (WGT) experiment to identify the genomic fragments causative to the observed tolerance. Surprisingly, careful comparison between the genomic sequences of the C. glabrata donor and S. cerevisiae recipient yeasts did not reveal any such fragment and only a few non-synonymous single nucleotide polymorphisms (SNPs) could be detected. The inventors could track down the tolerance to a mutant AST2 allele, more particularly to AST2^{N406I}. Ast2 also known as ATPase STabilizing2 (Chang and Fink 1995 J Cell Bio 128: 39-49) has been classified—based on sequence homology—as a member of the quinone oxidoreductase subgroup in the superfamily of medium-chain dehydrogenase/reductases (MDR) (Riveros-Rosas et al. 2003 EurJ Biochem 270: 3309-3334). AST2 has a close paralog, AST1, that arose from the whole genome duplication of S. cerevisiae. No studies have been reported on Ast2 and the art is completely silent about a link with tolerance towards HMT or other fermentation inhibitors present in lignocellulose hydrolysates.

Another surprising finding was that the causative AST2^{N406I} mutation did not originate from the donor gDNA by e.g. recombination between homologous sequences. This was also true for the other SNPs: none of the SNPs was present in the C. glabrata genomic DNA. Multiple controls have been performed excluding the possibility of the donor DNA acting as a random mutagen or a mutation inducing stress factor. However, the introduction of gDNA from a strain with higher tolerance appeared to be essential. Hence, the only explanation that can envisaged is that part of the foreign gDNA in some way transiently protects the host strain against the stress condition, allowing it to multiply during a few generations, creating more time and opportunity to generate spontaneous mutations that confer higher tolerance to the stress condition. This explains the very low number of SNPs detected and the presence of just a single causative AST2^{N406I} SNP, absent from the donor gDNA.

Another surprising finding herein disclosed is that although a search has been set up for HMF-tolerant yeasts, the obtained yeast strains expressing AST2^{N406I} not only tolerate HMF but also other inhibitors as furfural, acetic acid and vanillin. Hence, yeasts comprising the mutant AST2^{N406I} allele show improved fermentation efficiency in second generation substrates as well as in media spiked with fermentation inhibitors present in lignocellulosic hydrolysates. This is particularly interesting because poor acetic acid tolerance is also an important problem of yeast strains used in first generation bioethanol production (e.g. using molasses) because water recycling practices enhance acetic acid levels in the fermentations. Current application thus provides a solution to several industrially highly relevant problems.

In a first aspect, the protein depicted in SEQ ID No. 2 is provided. SEQ ID No. 2 depicts the Ast2 amino acid sequence of S. cerevisiae wherein the asparagine residue (N) on position 406 is replaced by isoleucine (I), said sequence is referred to herein as the Ast2^{N406I} mutant protein. Also the nucleic acid molecule encoding said protein is provided. Expressing the nucleic acid molecule in yeast provides the yeast with a tolerance to fermentation inhibitors HMF, furfural, formic acid and/or acetic acid.

In a second aspect, the application provides a chimeric gene comprising:
a promoter which is active in a eukaryotic cell,
a nucleic acid molecule encoding an amino acid sequence with a sequence identity of at least 90%, 91%, 92%, 93%, 94%, 95%, 96%, 97%, 98% or 99% to SEQ ID No. 1, said amino acid sequence comprises an isoleucine residue on position 406 of SEQ ID No. 1 or alternatively phrased comprises the 406I SNP, and
a 3′ end region involved in transcription termination or polyadenylation.

SEQ ID No. 1 depicts the S. cerevisiae yeast Ast2 protein (UniProtKB—P39945; https://www.yeastgenome.org/locus/5000000903). In one embodiment, said sequence identity to SEQ ID No. 1 is determined over the full range of 430 amino acids. It is clear that the amino acid sequence encoded by the disclosed chimeric genes should not be identically the same to SEQ ID No. 2 to still have the same effect. Indeed, the application discloses that from 1011 S. cerevisiae strains 8 strains comprise the 406I SNP, illustrating that the 406I SNP is causal to the features disclosed herein. Hence, a version with one or several additional mutations can still have the desired effect as long as the AST2 allele comprises the 406I SNP. In a particular embodiment, the above chimeric genes are provided wherein except for the N406I mutation, the sequence differences between the amino acid sequence and SEQ ID No. 1 are one or more selected from the list consisting of A3E, F185L, D274G, T286A, P346S and Y413Y. Sequence differences can also be attributed to conservative amino acid substitutions. Indeed, conservative amino acid substitutions refer to the interchangeability of residues having similar side chains. In a particular embodiment, the above chimeric genes are provided wherein the sequence differences between the amino acid sequence and SEQ ID No. 1 are exclusively related to conservative amino acid substitutions, except for the N406I mutation. Classes of amino acid residues for conservative substitutions are for example:

Acidic residues: Asp (D) and Glu (E)

Basic residues: Lys (K), Arg (R) and His (H)

Hydrophilic uncharged residues: Ser (S), Thr (T), Asn (N) and Gln (Q)

Aliphatic uncharged residues: Gly (G), Ala (A), Val (V), Leu (L) and Ilr (I)

Non-polar uncharged residues: Cys (C), Met (M) and Pro (P)

Aromatic residues: Phe (F), Tyr (Y) and Trp (W)

Alternative conservative amino acid residue substitution classes are class 1 (A, S and T), class 2 (D and E), class 3 (N and Q), class 4 (R and K), class 5 (1, L and M) and class 6 (F, Y and W).

Or alternatively, for example:

Alcohol group-containing residues: S and T

Aliphatic residues: I, L, V, and M

Cycloalkenyl-associated residues: F, H, W, and Y

Hydrophobic residues: A, C, F, G, H, I, L, M, R, T, V, W, and Y

Negatively charged residues: D and E

Polar residues: C, D, E, H, K, N, O R, S, and T

Positively charged residues: H, K, and R

Small residues: A, C, D, G, N, P, S, T, and V

Very small residues: A, G, and S

Residues involved in turn formation: A, C, D, E, G, H, K, N, Q, R, S, P and T

Flexible residues: Q, T, K, S, G, P, D, E, and R

In a particular embodiment, the above chimeric gene is provided wherein the nucleic acid molecule encodes the amino acid sequence of SEQ ID No. 2.

Throughout this application, any of the above described nucleic acid molecules, amino acid sequences and chimeric genes will be respectively referred to as any of the nucleic acid molecules, any of the amino acid sequences and any of the chimeric genes of the invention.

A “chimeric gene” or “chimeric construct” is a recombinant nucleic acid sequence in which a promoter or regulatory nucleic acid sequence is operably linked to, or associated with, a nucleic acid molecule that codes for a mRNA and encodes an amino acid sequence, such that the promoter is able to regulate transcription or expression of the associated nucleic acid coding sequence. The promoter of a chimeric gene is thus a heterologous promoter or alternatively phrased not the promoter which is operably linked to the associated nucleic acid sequence as found in nature. “Heterologous” as used herein applies to non-natural combinations of nucleic acid or amino acid sequences, i.e. combinations where at least two of the combined sequences (e.g. promoter and coding sequence) are foreign with respect to each other. Hence, in current application, the promoter of any chimeric gene of the invention is not the AST2 promoter or not the promoter which is naturally operably linked to the nucleic acid molecule encoding SEQ ID No. 1. or SEQ ID No. 2.

In a particular embodiment, the promoter in any of the chimeric gene of the invention is active in yeast. In a preferred embodiment, said promoter is selected from the list comprising pTEF1 (Translation Elongation Factor 1); pTEF2; pHXT1 (Hexose Transporter 1); pHXT2; pHXT3; pHXT4; pTDH3 (Triose-phosphate Dehydrogenase) also known in the art as pGADPH (Glyceraldehyde-3-phosphate dehydrogenase) or pGDP or pGLD1 or pHSP35 or pHSP36 or pSSS2; pTDH2 also known in the art as pGLD2; pTDH1 also known in the art as pGLD3; pADH1 (Alcohol Dehydrogenase) also know in the art as pADC1; pADH2 also known in the art as pADR2; pADH3; pADH4 also known in the art as pZRG5 or pNRC465; pADH5; pADH6 also known in the art as pADHVI; pPGK1 (3-Phosphoglycerate Kinase); pGAL1 (Galactose metabolism); pGAL2; pGAL3; pGAL4; pGAL5 also known in the art as pPGM2 (Phosphoglucomutase); pGAL6 also known in the art as pLAP3 (Leucine Aminopeptidase) or pBLH1 or pYCP1; pGAL7; pGAL10; pGAL11 also known in the art as pMED15 or pRAR3 or pSDS4 or SPT13 or ABE1; pGAL80; pGAL81; pGAL83 also know in the art as pSPM1; pSIP2 (SNF1-interacting Protein) also know in the art as pSPM2; pMET (Methionine requiring); pPMA1 (Plasma Membrane ATPase) also known in the art as pKTI10; pPMA2; pPYK1 (Pyruvate Kinase) also known in the art as pCDC19; pPYK2; pENO1 (Enolase) also known in the art as pHSP48; pENO2; pPHO (Phosphate metabolism); pCUP1 (Cuprum); pCUP2 also known in the art as pACE1; pPET56 also known in the art as pMRM1 (Mitochondria) rRNA Methyltransferase); pNMT1 (N-Myristoyl Transferase) also known in the art as pCDC72; pGRE1 (Genes de Respuesta a Estres); pGRE2; GRE3; pSIP18 (Salt Induced Protein); pSV40 (Simian Vacuolating virus) and pCaMV (Cauliflower Mosaic Virus). These promoters are widely used in the art. The skilled person will have no difficulty identifying them in databases. For example, the skilled person will consult the Saccharomyces genome database website (http://www.yeastgenome.org/) or the Promoter Database of Saccharomyces cerevisiae (http://rulai.cshl.edu/SCPD/) for retrieving the yeast promoters' sequences. Yeast, as used here, can be any yeast useful for industrial applications. In a particular embodiment, said yeast is useful for ethanol production, including, but not limited to Saccharomyces, Zygosaccharomyces, Brettanomyces and Kluyveromyces. More particularly, said yeast is a Saccharomyces sp., even more preferably it is a Saccharomyces cerevisiae sp. In another particular embodiment, said yeast is a xylose fermenting yeast or a second-generation yeast or a yeast able to ferment lignocellulose hydrolysates.

In another aspect a vector is provided comprising any of the chimeric genes of the invention. The term “vector” refers to any linear or circular DNA construct. The vector can refer to an expression cassette or any recombinant expression system for the purpose of expressing a nucleic acid sequence of the invention in vitro or in vivo, constitutively or inducibly, in any cell, including yeast cells. The vector can remain episomal or integrate into the host cell genome. The vector can have the ability to self-replicate or not (i.e. drive only transient expression in a cell). The term includes recombinant expression cassettes that contain only the minimum elements needed for transcription of the recombinant nucleic acid. The vector of the invention is a “recombinant vector” which is by definition a man-made vector.

AST1^{D405I} Alleles and Chimeric Genes Comprising them

The application also discloses a novel and inventive mutant allele of the AST1 yeast gene. AST1 is a paralog of AST2 (see above). More particularly the application provides the AST1^{D405I} allele, i.e. a nucleic acid molecule encoding an amino acid sequence with a sequence identity of at least 90%, 91%, 92%, 93%, 94%, 95%, 96%, 97%, 98% or 99% to SEQ ID No. 3, said amino acid sequence comprises an isoleucine residue on position 405 of SEQ ID No. 3. SEQ ID No. 3 depicts the S. cerevisiae yeast Ast1 protein (UniProtKB—P35183; https://www.yeastgenome.org/locus/5000000165). In one embodiment, a nucleic acid molecule encoding SEQ ID No. 4 is provided. SEQ ID No. 4 depicts the Ast1 amino acid sequence of S. cerevisiae wherein the aspartate residue (D) on position 405 is replaced by isoleucine (I), said sequence is referred to herein as the Ast1^{D405I} mutant protein. Expressing the AST1^{D405I} mutant allele in yeast that comprises a mutant AST2^{N406I} allele (see above) further increases the tolerance to fermentation inhibitors HMF, furfural, formic acid or acetic acid. The AST1^{D405I} mutation can be engineered in yeast by gene editing, for example by the well-known Crispr-Cas9 technology or can be introduced as a chimeric gene. Therefore, the application provides a chimeric gene comprising:
a promoter which is active in a eukaryotic cell,
a nucleic acid molecule encoding an amino acid sequence with a sequence identity of at least 90%, 91%, 92%, 93%, 94%, 95%, 96%, 97%, 98% or 99% to SEQ ID No. 3, said amino acid sequence comprises an isoleucine residue on position 405 of SEQ ID No. 3, and
a 3′ end region involved in transcription termination or polyadenylation.

In one embodiment, said sequence identity is determined over the full range of 429 amino acids. In a particular embodiment, the above chimeric gene is provided wherein the sequence differences between the amino acid sequence and SEQ ID No. 3 are exclusively related to conservative amino acid substitutions, except for the D405I mutation.

Engineered Yeast Strains to Overcome Fermentation Inhibition

In a fourth aspect, a yeast is provided comprising any of the nucleic acid molecules, amino acid sequences or chimeric genes of the invention.

In one embodiment, a xylose fermenting yeast is provided comprising an amino acid sequence with sequence identity of at least 90%, 91%, 92%, 93%, 94%, 95%, 96%, 97%, 98% or 99% to SEQ ID No. 1, said amino acid sequence comprises an isoleucine residue on position 406 of SEQ ID No. 1. Also a yeast or a xylose fermenting yeast is provided being able to grow and metabolize lignocellulosic hydrolysates comprising one or more growth inhibiting compounds, wherein said yeast comprises an amino acid sequence with sequence identity of at least 90%, 91%, 92%, 93%, 94%, 95%, 96%, 97%, 98% or 99% to SEQ ID No. 1, said amino acid sequence comprises an isoleucine residue on position 406 of SEQ ID No. 1. In a particular embodiment, the sequence differences between the amino acid sequence and SEQ ID No. 1 are, besides the 406I SNP, one or more selected from the list consisting of A3E, F185L, D274G, T286A, P346S and Y413Y and/or related to conservative amino acid substitutions. In a particular embodiment, said one or more growth inhibiting compounds are selected from the list consisting of hydroxymethylfurfural (HMF), furfural, formic acid, acetic acid, levulinic acid, 4-hydroxybenzoic acid, 4-hydroxybenzaldehyde and vanillin. In a more particular embodiment, said one or more growth inhibiting compounds are HMF, furfural, formic acid and/or acetic acid. Hydroxymethylfurfural is also known as 5-(hydroxymethyl)furfural.

In another particular embodiment, said yeast is an ethanol producing yeast being able to grow and produce ethanol from lignocellulosic hydrolysates comprising one or more growth inhibiting compounds selected from the list consisting of HMF, furfural, formic acid, acetic acid, levulinic acid, 4-hydroxybenzoic acid, 4-hydroxybenzaldehyde and vanillin, wherein said yeast strain comprises a nucleic acid molecule encoding SEQ ID No. 2 or alternatively phrased comprises an AST2^{N406I} allele. In another particular embodiment, said yeast is an industrial yeast, an ethanol producing yeast, a second-generation yeast and/or a xylose-fermenting yeast. In a most particular embodiment, said yeast is not the wine yeast CBS5835, not EXF7145 (a natural isolate from oak), not NCYC3985 (a natural isolate from wax on rock surface), not Lib 73 (an isolate from grape must), not CLIB564 or CLIB558 (two isolates from dairy cheese camembert), not CBS2421 (an isolate from Japanese kefir grains) or not EN14S01 (a soil isolate from Taiwan).

Also provided is a culture comprising second-generation substrates or lignocellulosic hydrolysates and any of the above described yeasts.

In another embodiment, an ethanol producing yeast is provided for reducing the production of acetaldehyde in a yeast fermentation, the yeast comprising a nucleic acid molecule encoding an amino acid sequence with a sequence identity of at least 90%, 91%, 92%, 93%, 94%, 95%, 96%, 97%, 98% or 99% to SEQ ID No. 1, said amino acid sequence comprises an isoleucine residue on position 406 of SEQ ID No. 1. In a particular embodiment, the sequence differences between the amino acid sequence and SEQ ID No. 1 are, besides the 406I SNP, one or more selected from the list consisting of A3E, F185L, D274G, T286A, P346S and Y413Y and/or related to conservative amino acid substitutions. Reducing the production of acetaldehyde means a statistically significant reduction of the acetaldehyde production compared to a control yeast, i.e. a yeast not comprising an AST2^{N406I} allele. In a particular embodiment, said yeast fermentation in which the production of acetaldehyde is reduced is a beer or wine fermentation. In another particular embodiment, an alcoholic beverage (more particularly beer or wine) is provided, said beverage is produced by a method comprising the step of adding one of the above described ethanol producing yeasts to a wort or most.

Also provided is a culture comprising wort or most and comprising an ethanol producing yeast comprising a nucleic acid molecule encoding an amino acid sequence with a sequence identity of at least 90%, 91%, 92%, 93%, 94%, 95%, 96%, 97%, 98% or 99% to SEQ ID No. 1, said amino acid sequence comprises an isoleucine residue on position 406 of SEQ ID No. 1. In a particular embodiment, the sequence differences between the amino acid sequence and SEQ ID No. 1 are, besides the 406I SNP, one or more selected from the list consisting of A3E, F185L, D274G, T286A, P346S and Y413Y and/or related to conservative amino acid substitutions. In a most particular embodiment, said yeast is not CBS5835.

In another embodiment, said one of the herein described yeasts is a genetically engineered or a recombinant yeast strain, engineered for the fermentation of second-generation substrates or for the production of second-generation biofuels and/or bio-based compounds or for the production of alcoholic beverages as beer or wine with reduced acetaldehyde levels. Genetic engineering comprises the transformation of yeast with recombinant vectors comprising chimeric genes but is not restricted to that. Genetic engineering also comprises the use of the gen(om)e editing technology such as the CRISPR-Cas system. CRISPR interference is a genetic technique which allows for sequence-specific control of gene expression in prokaryotic and eukaryotic cells. It is based on the bacterial immune system-derived CRISPR (clustered regularly interspaced palindromic repeats) pathway and has been modified to edit basically any genome. By delivering the Cas nuclease (in many cases Cas9) complexed with a synthetic guide RNA (gRNA) in a cell, the cell's genome can be cut at a desired location depending on the sequence of the gRNA, allowing subtly removing, replacing or inserting single nucleotides (e.g. DiCarlo et al 2013 Nucl Acids Res doi:10.1093/nar/gkt135; Sander & Joung 2014 Nat Biotech 32:347-355). In one particular embodiment, the engineered yeast strain of the application is engineered by making use of the Crispr/Cas technology.

In a particular embodiment, any of the yeasts described above is provided further comprising a nucleic acid molecule encoding an amino acid sequence with sequence identity of at least 90%, 91%, 92%, 93%, 94%, 95%, 96%, 97%, 98% or 99% to SEQ ID No. 3, said amino acid sequence comprises an isoleucine residue on position 405 of SEQ ID No. 3 or further comprising a nucleic acid molecule encoding SEQ ID No. 4.

In one embodiment an enriched culture of one of the yeast strains of current application is provided. The term “culture” as used herein refers to a population of microorganisms that are propagated on or in media of various kinds. An “enriched culture” of one of the yeast strains of current application refers to a yeast culture wherein the total yeast population of the culture contains more than 50%, more than 60%, more than 70%, more than 80%, more than 90%, or more than 95% of one of the yeast strains of current application. This is equivalent as saying that a yeast culture is provided, wherein said culture is enriched with one of the yeast strains of current application and wherein “enriched” means that the total yeast population of said culture contains more than 50%, more than 60%, more than 70%, more than 80%, more than 90%, or more than 95% of one of the yeast strains of current application.

In another embodiment, a biologically pure culture of one of the yeast strains of current application is provided. As used herein, “biologically pure” refers to a culture which contains substantially no other microorganisms than the desired strain of microorganism and thus a culture wherein virtually all of the cells present are of the selected strain. In practice, a culture is defined biologically pure if the culture contains at least more than 96%, at least more than 97%, at least more than 98% or at least more than 99% of one of the yeast strains of current application. When a biologically pure culture contains 100% of the desired microorganism a monoculture is reached. A monoculture thus only contains cells of the selected strain and is the most extreme form of a biologically pure culture.

### Methods and Uses to Improve Fermentation Efficiency

The Applicant report that the 406I SNP in the yeast Ast2 protein relates to increased tolerance towards fermentation inhibitors and to reduced production of acetaldehyde. Therefore, in another aspect, any of the chimeric genes or any yeast strains of the invention can be used for obtaining or increasing tolerance towards fermentation inhibitors or for reducing the production of acetaldehyde in a yeast or yeast culture. The use is thus provided of the AST2^{N406I} SNP or of any of the chimeric genes of the invention or of any of the vectors herein described for obtaining or increasing tolerance towards fermentation inhibitors in a eukaryotic organism. The use is also provided of the AST2^{N406I} SNP or of any of the yeast strains herein described for reducing the production of acetaldehyde in a yeast culture, particularly in an alcoholic beverage fermentation.

“Obtaining tolerance” or “increasing tolerance” as used herein means that the yeast cell that comprises the AST2^{N406I} SNP or any of the nucleic acid sequences, chimeric genes or vectors of the invention shows less of an effect (statistically significant with p-value<0.05), or no effect, compared to a corresponding reference yeast cell lacking the SNP, nucleic acid sequence, chimeric gene or vector of the invention in response to the presence of compound levels that have an inhibitory effect on the said reference yeast cell. For the current application, said compound is one of the group consisting of HMF, furfural, formic acid, acetic acid, levulinic acid, 4-hydroxybenzoic acid, 4-hydroxybenzaldehyde and vanillin. This effect can be related to growth, proliferation or metabolic activity of the organism. Preferably, for yeast, increasing tolerance is achieved when a yeast strain comprising any of the nucleic acid sequences, chimeric genes or vectors of the invention is still actively dividing or metabolically active in the fermentation process in contrast to the control strain lacking the nucleic acid, chimeric gene or vector of the invention. This effect can be convincingly measured by using the optical density or absorbance of a sample of the yeast culture at a wavelength of 600 nm also referred to in the art as OD600. More preferably, the OD600 of the tolerant yeast strain comprising the nucleic acid sequence, chimeric gene or vector of the invention would preferably at least be 20%, preferably at least be 30%, more preferably at least be 40%, more preferably at least be 50%, even more preferably at least be 60%, even more preferably at least be 70%, even more preferably at least be 80%, even more preferably at least be 90%, and most preferably at least be 100% higher compared to a control strain lacking the nucleic acid sequence of the invention at growth limiting levels for the said control strain. The metabolic activity can also be measured by the production of ethanol, for example with gas chromatography (GC).

Levels of fermentation inhibitors that reduce the fermentation efficiency can be defined as those levels of the yeast substrate that inhibit or at least negatively influence the growth, proliferation or metabolic activity of yeast cells with a statistically significant difference (p<0.05) or with at least 5%, 10%, 15%, 20%, 30%, 40%, 50%, 60%, 70%, 80%, 90% compared to the growth, proliferation or metabolic activity of yeast cells on a substrate optimized for fermentation, preferably industrial fermentation. The production of metabolites as output of “metabolic activity” can be convincingly measured by high performance liquid chromatography (HPLC). In particular embodiments, the level of HMF, furfural, formic acid or acetic acid that reduce fermentation efficiency is 0.25 g/l or more, 0.5 g/l or more, 0.75 g/l or more, 1 g/l or more, 2 g/l or more, or 5 g/l or more, or 6 g/l or more, or 7 g/l or more, or more particularly for HMF and formic acid between 2 and 12 g/l and for furfural and acetic acid between 0.5 and 10 g/l. These levels are levels that inhibit fermentation efficiency in yeast (see Example 1) when spiked in lignocellulosic hydrolysates. These hydrolysates intrinsically comprise HMF, furfural, formic acid and/or acetic acid as well. Hence, the levels of HMF, furfural, formic acid and/or acetic acid that inhibit fermentation capacity of yeast are lower. In particular embodiments, the levels of HMF, furfural, formic acid or acetic acid that inhibit fermentation efficiency in yeast are between 0.1 and 5 g/l or between 0.15 and 8 g/l or between 0.2 and 10 g/l.

A method for obtaining or increasing tolerance in yeasts towards fermentation inhibitors selected from the group consisting of hydroxymethylfurfural, furfural, formic acid, acetic acid, levulinic acid, 4-hydroxybenzoic acid, 4-hydroxybenzaldehyde and vanillin is provided, the method comprising the step of replacing the amino acid residue on position 406 of SEQ ID No. 1 by isoleucine or the step of introducing any of the chimeric genes of the invention in said yeast.

Another aspect of the invention relates to a process of producing a fermentation product selected from the group consisting of ethanol, lactic acid, 3-hydroxy-propionic acid, acrylic acid, acetic acid, succinic acid, citric acid, amino acids, 1,3-propane-diol, ethylene, glycerol, butyric acid, caproate, butanol, glyoxylate, muconic acid, fatty alcohols, fatty acids, β-lactam antibiotics and cephalosporins. The process preferably comprises the steps of: a) fermenting a medium comprising a carbon source and one or more growth inhibiting compounds selected from the group consisting of hydroxymethylfurfural, furfural, formic acid, acetic acid, levulinic acid, 4-hydroxybenzoic acid, 4-hydroxybenzaldehyde and vanillin, wherein the yeast ferments the carbon source to the fermentation product and optionally, b) recovery of the fermentation product. In a particular embodiment, said yeast comprises a nucleic acid molecule encoding an amino acid sequence with a sequence identity of at least 90%, 91%, 92%, 93%, 94%, 95%, 96%, 97%, 98% or 99% to SEQ ID No. 1, said amino acid sequence comprises an isoleucine residue on position 406 of SEQ ID No. 1. or said yeast is any one of the yeast strains of the invention. In another particular embodiment, the sequence differences between the amino acid sequence and SEQ ID No. 1 are, besides the 406I SNP, one or more selected from the list consisting of A3E, F185L, D274G, T286A, P346S and Y413Y and/or related to conservative amino acid substitutions. In a most particular embodiment, said yeast is not CBS5835. In yet another particular embodiment, said medium comprising a carbon source is a second-generation substrate or a lignocellulosic hydrolysate.

A preferred fermentation process according to the invention is a process for the production of ethanol, whereby the process comprises the steps of: a) fermenting a medium comprising a source of xylose and one or more growth inhibiting compounds selected from the group consisting of hydroxymethylfurfural, furfural, formic acid, acetic acid, levulinic acid, 4-hydroxybenzoic acid, 4-hydroxybenzaldehyde and vanillin with any of the yeasts of the invention, whereby the yeast ferments xylose, and optionally, b) recovering the produced ethanol. The fermentation process may further be performed as described above. In the process the volumetric ethanol productivity is preferably at least 0.5, 1.0, 1.5, 2.0, 2.5, 3.0, 5.0 or 10.0 g ethanol per litre per hour. The ethanol yield on xylose and/or glucose in the process preferably is at least 50, 60, 70, 80, 90, 95 or 98%. The ethanol yield is herein defined as a percentage of the theoretical maximum yield, which, for xylose and glucose is 0.51 g. ethanol per g. xylose or glucose.

In another aspect, a method to produce an alcoholic beverage is provided comprising the steps of adding a yeast strain to a fermentation medium in conditions allowing the yeast to produce the alcoholic beverage, said yeast strain comprises a nucleic acid molecule encoding an amino acid sequence with a sequence identity of at least 90%, 91%, 92%, 93%, 94%, 95%, 96%, 97%, 98% or 99% to SEQ ID No. 1, said amino acid sequence comprises an isoleucine residue on position 406 of SEQ ID No. 1, or said yeast is any one of the yeast strains of the invention. In another particular embodiment, the sequence differences between the amino acid sequence and SEQ ID No. 1 are, besides the 406I SNP, one or more selected from the list consisting of A3E, F185L, D274G, T286A, P346S and Y413Y and/or related to conservative amino acid substitutions. In a most particular embodiment, said yeast is not CBS5835. In an even more particular embodiment, said yeast comprises a nucleic acid molecule encoding SEQ ID No. 2.

In a particular embodiment, said alcoholic beverage has a statistically significant reduced level of acetaldehyde compared to an alcoholic beverage produced by a control yeast in the same conditions. A control yeast is a genetically identical yeast but does not comprise any of the nucleic acid molecules of the invention. In another particular embodiment, said beverage is beer or wine. In a most particular embodiment, said yeast is not CBS5835. The application thus also provides methods to reduce the production of acetaldehyde in an ethanol producing yeast fermentation comprising the steps of providing a fermentation medium for the production of ethanol of for the production of an alcoholic beverage such as beer or wine; adding one or more yeast strains to the fermentation medium, wherein at least one of the yeast strains is a yeast comprising a nucleic acid molecule encoding an amino acid sequence with a sequence identity of at least 90%, 91%, 92%, 93%, 94%, 95%, 96%, 97%, 98% or 99% to SEQ ID No. 1, said amino acid sequence comprises an isoleucine residue on position 406 of SEQ ID No. 1, in particular wherein the sequence differences between the amino acid sequence and SEQ ID No. 1 are, besides the 406I SNP, one or more selected from the list consisting of A3E, F185L, D274G, T286A, P346S and Y413Y and/or related to conservative amino acid substitutions, or a yeast comprising a nucleic acid molecule encoding SEQ ID No. 2 or comprising any of the nucleic acid molecules or chimeric genes of the invention; optionally measuring the acetaldehyde in the produced ethanol or alcoholic beverage and optionally concluding that a reduced level of acetaldehyde is produced when a statistically significant lower level (p<0.05) of acetaldehyde is present compared to ethanol or alcoholic beverage produced by a control yeast strain.

The carbon source used in any of the fermentation methods described herein can be a source of xylose or of glucose or of any other type of carbohydrate such as e.g. in particular a source of arabinose. The sources of xylose and glucose may be xylose and glucose as such (i.e. as monomeric sugars) or they may be in the form of any carbohydrate oligo- or polymer comprising xylose and/or glucose units, such as e.g. lignocellulose, xylans, cellulose, starch and the like. For release of xylose and/or glucose units from such carbohydrates, appropriate carbohydrases (such as xylanases, glucanases, amylases, cellulases, glucanases and the like) may be added to the fermentation medium or may be produced by the modified host cell. In the latter case the modified host cell may be genetically engineered to produce and excrete such carbohydrases. An additional advantage of using oligo- or polymeric sources of glucose is that it enables to maintain a low(er) concentration of free glucose during the fermentation, e.g. by using rate-limiting amounts of the carbohydrases preferably during the fermentation. This, in turn, will prevent repression of systems required for metabolism and transport of non-glucose sugars such as xylose. In a preferred process the modified host cell ferments both the xylose and glucose, preferably simultaneously in which case preferably a modified host cell is used which is insensitive to glucose repression to prevent diauxic growth. In addition to a source of xylose (and glucose) as carbon source, the fermentation medium will further comprise the appropriate ingredient required for growth of the modified host cell. Compositions of fermentation media for growth of eukaryotic microorganisms such as yeasts are well known in the art. In a particular embodiment, said medium comprising a carbon source is a second-generation substrate or a lignocellulosic hydrolysate.

Any of the fermentation processes herein disclosed may be an aerobic or an anaerobic fermentation process. An anaerobic fermentation process is herein defined as a fermentation process run in the absence of oxygen or in which substantially no oxygen is consumed, preferably less than 5, 2.5 or 1 mmol/l/h, more preferably 0 mmol/l/h is consumed (i.e. oxygen consumption is not detectable), and wherein organic molecules serve as both electron donor and electron acceptors. In the absence of oxygen, NADH produced in glycolysis and biomass formation, cannot be oxidised by oxidative phosphorylation. To solve this problem many microorganisms use pyruvate or one of its derivatives as an electron and hydrogen acceptor thereby regenerating NAD⁺. Thus, in a preferred anaerobic fermentation process pyruvate is used as an electron (and hydrogen acceptor) and is reduced to fermentation products such as ethanol, as well as non-ethanol fermentation products such as lactic acid, 3-hydroxy-propionic acid, acrylic acid, acetic acid, succinic acid, citric acid, amino acids, 1,3-propane-diol, ethylene, glycerol, butyric acid, caproate, butanol, glyoxylate, muconic acid, fatty alcohols, fatty acids, β-lactam antibiotics and cephalosporins. Anaerobic processes of the invention are preferred over aerobic processes because anaerobic processes do not require investments and energy for aeration and in addition, anaerobic processes produce higher product yields than aerobic processes. Alternatively, the fermentation process of the invention may be run under aerobic oxygen-limited conditions. Preferably, in an aerobic process under oxygen-limited conditions, the rate of oxygen consumption is at least 5.5, more preferably at least 6 and even more preferably at least 7 mmol/l/h.

Any of the fermentation processes described above is preferably run at a temperature that is optimal for any of the yeasts of the invention. Thus, for most yeasts, the fermentation process is performed at a temperature which is less than 42° C., preferably less than 38° C. For yeast, the fermentation process is preferably performed at a temperature which is lower than 35, 33, 30 or 28° C. and at a temperature which is higher than 20, 22, or 25° C. For some species, such as Kluyveromyces marxianus, and engineered Saccharomyces cerevisiae strains, the fermentation process may be run at considerably higher temperatures, i.e. at 42° C., 43° C., or preferably between 45 and 50° C., or in rare cases between 50 and 55° C.

In a final aspect, the application provides a method of producing a yeast strain for tolerating the presence of a growth inhibiting level of one or more fermentation inhibitors selected from the list consisting of hydroxymethylfurfural, furfural, formic acid, acetic acid, levulinic acid, 4-hydroxybenzoic acid, 4-hydroxybenzaldehyde and vanillin, more particularly a growth inhibiting level of HMF, furfural, formic acid and/or acetic acid. The growth inhibiting levels are those that are described earlier in current application. “For tolerating” is the same as “able to tolerate” and refers to a statistically significant increased level of tolerance to one of said fermentation inhibitors. The application also provides a method of producing a yeast strain with a statistically significantly increased tolerance (p<0.05) to said level of said inhibitors and a method of producing a yeast for a statistically significantly reduced acetaldehyde production (p<0.05). Said methods of the final aspect comprise the step of expressing at least one nucleic acid molecule encoding an amino acid sequence with a sequence identity of at least 90%, 91%, 92%, 93%, 94%, 95%, 96%, 97%, 98% or 99% to SEQ ID No. 1, said amino acid sequence comprises an isoleucine residue on position 406 of SEQ ID No. 1, more particularly wherein the sequence differences between the amino acid sequence and SEQ ID No. 1 are, besides the 406I SNP, one or more selected from the list consisting of A3E, F185L, D274G, T286A, P346S and Y413Y and/or related to conservative amino acid substitutions or even more particularly wherein the expressed nucleic acid molecule encodes SEQ ID No. 2. Said expression can be obtained by a genetic engineering step whereby any of the chimeric genes of the invention is introduced in the yeast according to methods well known by the skilled person. Said expression can as well be obtained by a gene editing step whereby the amino acid residue for example N on position 406 of SEQ ID No. 1 is replaced by 1.

It is to be understood that although particular embodiments, specific configurations as well as materials and/or molecules, have been discussed herein for cells and methods according to the present invention, various changes or modifications in form and detail may be made without departing from the scope and spirit of this invention. The following examples are provided to better illustrate particular embodiments, and they should not be considered limiting the application. The application is limited only by the claims.

### EXAMPLES

### Example 1. Analysis of the Major Inhibitors in Lignocellulose Hydrolysates

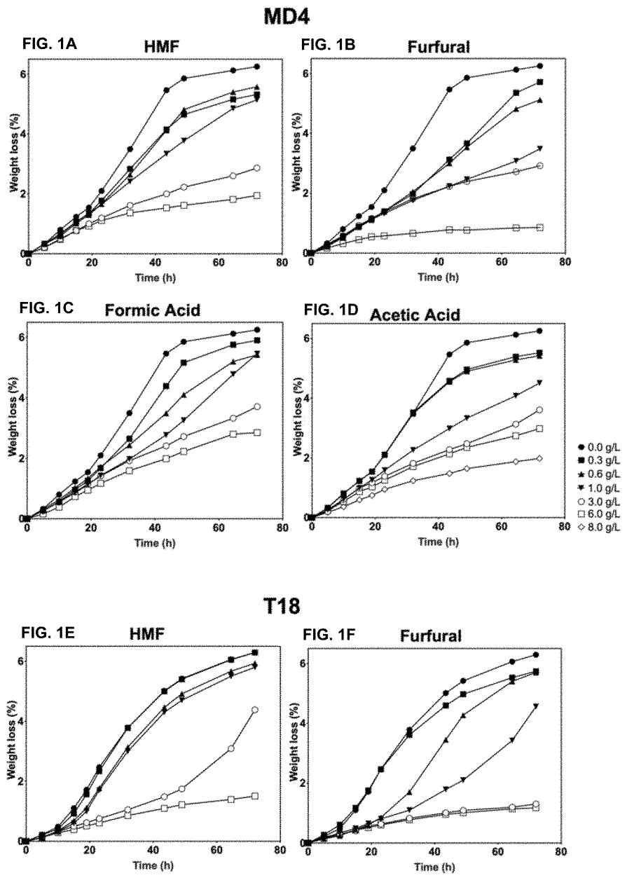

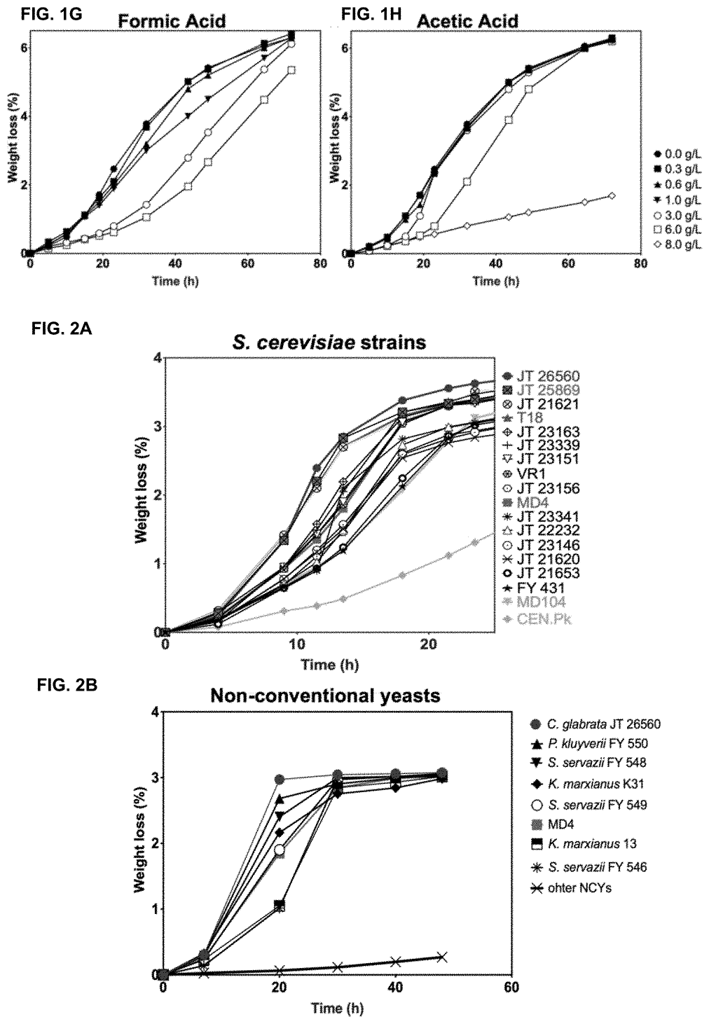

As a first step in developing a yeast strain that can cope with the presence of fermentation inhibitors, the level of all relevant inhibitory compounds was measured in five different lignocellulose hydrolysates: two bagasse hydrolysates, two corn cob hydrolysates and one spruce hydrolysate (for composition see Table 2). Fermentations with two 2G industrial yeast strains, MD4 and T18, were performed using these hydrolysates spiked with a range of inhibitor concentrations. For both strains, HMF, furfural, formic acid and acetic acid were identified as the major inhibitors. FIGS. 1A-1H shows the results obtained for one of the corn cob hydrolysates, but similar results were obtained for the other hydrolysates (data not shown). Levulinic acid, 4-hydroxybenzoic acid, 4-hydroxybenzaldehyde and vanillin had a smaller inhibitory effect under the tested conditions (data not shown).

### Example 2. Screening a Yeast Collection for High HMF Tolerance

Based on the data from Example 1, HMF was selected as representative for fermentation inhibitors present in lignocellulosic hydrolysates. Therefore, in a next step a screening was set up to identify HMF tolerant strains. The screened collection consisted of 2526 S. cerevisiae strains, as well as 17 non-conventional yeast species previously reported as displaying high tolerance to HMF during growth on solid nutrient medium (Mukherjee et al. 2017 Biotechnol Biofuels 10: 216). The screen was performed using solid nutrient medium with a high HMF concentration (8 g/l). 17 S. cerevisiae strains and four non-conventional yeast species with very high HMF tolerance were identified. These strains, as well as three industrial 2G S. cerevisiae strains, MD4, T18 and MD104, and the lab strain CEN.PK as control, were subsequently evaluated for HMF tolerance (8 g/l) in small-scale semi-anaerobic fermentations with synthetic medium (FIGS. 2A and 2B). A Candida glabrata strain (JT26560) was identified as the most HMF tolerant strain of all strains evaluated (FIGS. 2A and 2B). The 2G industrial S. cerevisiae strains, MD4, T18 and MD104, but not the lab strain CEN.PK, as well as three non-conventional yeast species, P. kluyveri, K. marxianus and S. servazii displayed similarly high HMF tolerance as the most HMF-tolerant S. cerevisiae strains identified in our collection.

### Example 3. Isolation of HMF-Tolerant Transformants of MD4 Using WGT with C. glabrata gDNA

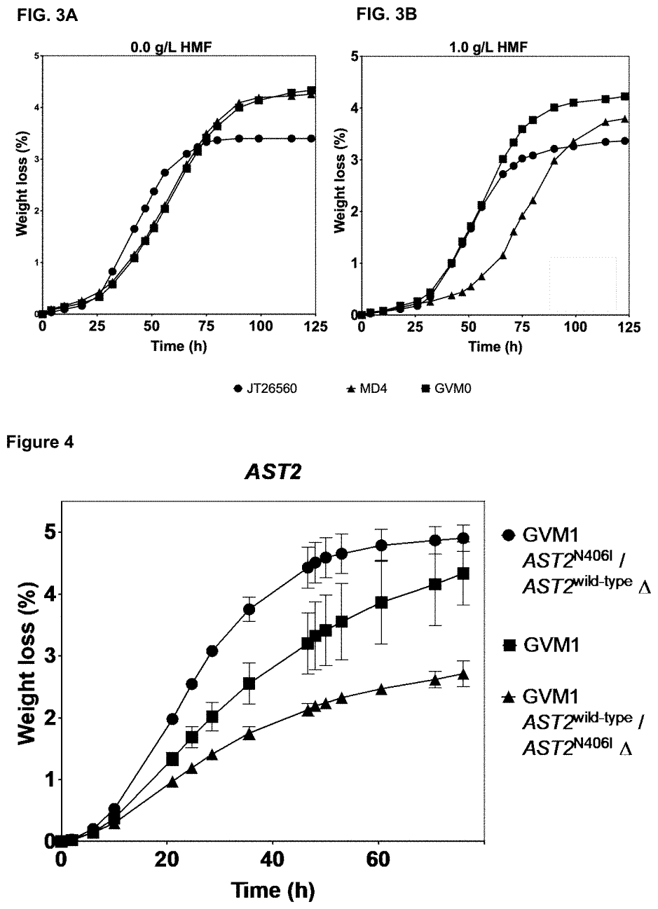

We have performed whole genome transformation (WGT) of the 2G industrial yeast strain MD4 using genomic DNA (gDNA) from the most HMF-tolerant strains identified in Example 2, i.e. the C. glabrata JT26560, four HMF-tolerant S. cerevisiae strains, the three other non-conventional yeast species with highest HMF tolerance (P. kluyveri, K. marxianus and S. servazii), five non-HMF-tolerant S. cerevisiae strains, as well as the recipient host strain MD4 itself. The transformants were selected on solid YPDX plates with 2.5 g/l HMF. Only when using gDNA of C. glabrata, we were able to obtain stable MD4 transformant strains displaying improved HMF tolerance both after restreaking on solid synthetic YPDX plates with 2.5 g/l or 4.0 g/l HMF, in small-scale semi-anaerobic fermentations with YPDX medium and 6 g/l or 12 g/l HMF and in corn cob hydrolysate enriched with 0.6 g/l, 1.0 g/l or 3.0 g/l HMF. Fermentations in HMF-enriched corn cob hydrolysate are shown in FIG. 3B for the MD4 recipient host strain, the C. glabrata gDNA donor strain and the transformant strain GVM0, which displayed the highest HMF tolerance of all transformants. The higher fermentation yield of strain GVM0 in corn cob hydrolysate spiked with 1 g/l HMF is probably due to its capacity to ferment xylose and possibly also due to higher ethanol tolerance compared to C. glabrata. The GVM0 transformant strain showed clearly improved HMF tolerance compared to the recipient host strain MD4 in corn cob hydrolysate spiked with 1 g/L HMF (FIG. 3B). In the absence of spiked HMF, the transformant strain GVM0 and the recipient host strain MD4 displayed the same fermentation performance in corn cob hydrolysate, indicating the absence of negative-side effects at least under this condition (FIG. 3A).

### Example 4. Identification of the Allele Causative for Enhanced HMF Tolerance

The tetraploid WG transformant, GVM0, was sporulated and a diploid segregant, GVM1, was isolated that showed identical fermentation performance in YPDX medium and corn cob hydrolysate spiked with 1 g/l HMF compared to the parent strain GVM0 (data not shown). Subsequently, MD4 and GVM1 were submitted to whole-genome sequence analysis. Bio-informatics analysis revealed only nine non-synonymous single nucleotide polymorphisms (SNPs) between both strains, as well as multiple synonymous SNPs. The nine non-synonymous SNPs were introduced in different chromosomes. Interestingly, all non-synonymous SNPs were absent in the genome of the C. glabrata gDNA donor strain used for WGT and were present in heterozygous form in strain GVM1.

Next, reciprocal hemizygosity analysis (RHA) was performed to identify which SNP(s) were responsible for the enhanced HMF tolerance in strain GVM1. For each of the nine genes, the mutant or the wild type allele was deleted in the diploid strain GVM1. In addition, other candidate genes with SNPs in the promotor or terminator region were investigated for a possible causative role in HMF tolerance. Only for the AST2 gene an effect was observed: deletion of the mutant AST2^{N406I} allele reduced HMF tolerance, while deletion of the wild type AST2 allele further enhanced HMF tolerance (FIG. 4). For the other SNPs evaluated, no difference in fermentation capacity in the presence of HMF between the hemizygous RHA strains containing either the mutant allele or the wild type allele was observed (data not shown).

### Example 5. Effect of AST2^{N406I} on Tolerance to Multiple Inhibitors in YPDX and in Corn Cob Hydrolysate

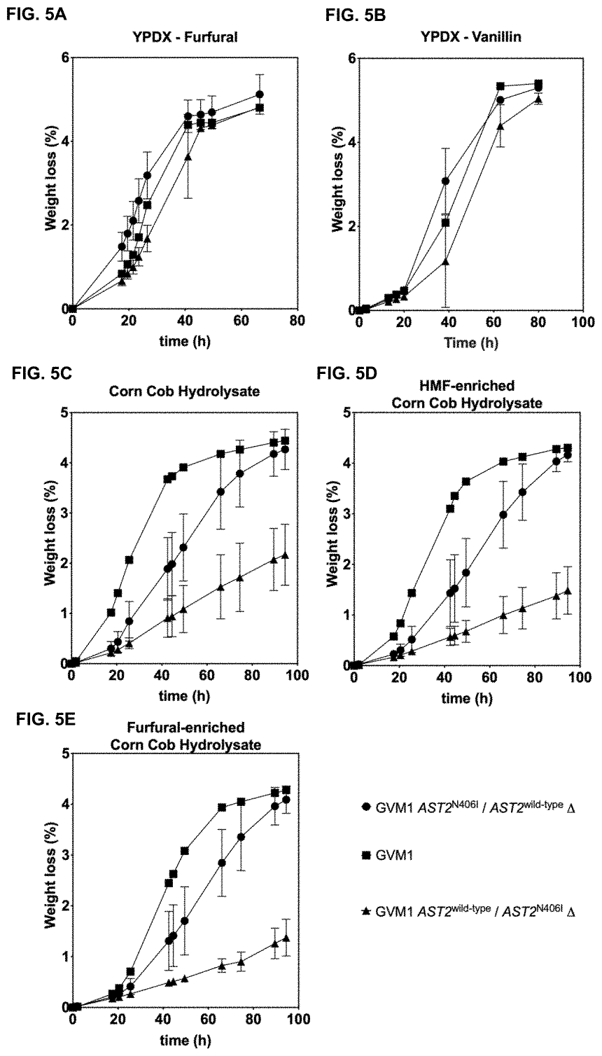

The hemizygous RHA strains of GVM1 expressing either the mutant or wild type AST2 allele were evaluated for tolerance to other inhibitors in comparison with the parent GVM1 strain. The results showed that in YPDX medium AST2^{N406I} compared to the wild type AST2 allele significantly improved tolerance to 4.0 g/l furfural and to a smaller extent also to 4.5 g/l vanillin (FIG. 5A-B). The GVM1 strain, comprising both the AST2^{N406I} and wild type AST2 allele, showed an intermediate performance. Interestingly, in corn cob hydrolysate, the strain expressing the AST2^{N406I} allele also showed a much better fermentation rate and yield compared to the strain with only the wild type AST2 allele. In this case, however, the GVM1 strain clearly showed the very best performance (FIG. 5C). When the corn cob hydrolysate was spiked with 1.0 g/l HMF or 1.0 g/l furfural, the fermentation performance significantly dropped in the RHA strain with the wild type AST2 allele only, while it was only slightly compromised in the GVM1 strain and the RHA strain expressing only AST2^{N406I} (FIG. 5D-E). The observation that the GVM1 strain showed the best fermentation performance in corn cob hydrolysate but not in YPDX medium indicates that in corn cob hydrolysate other factors besides HMF and furfural, that affect the fermentation, have a different dependency on AST2 activity.

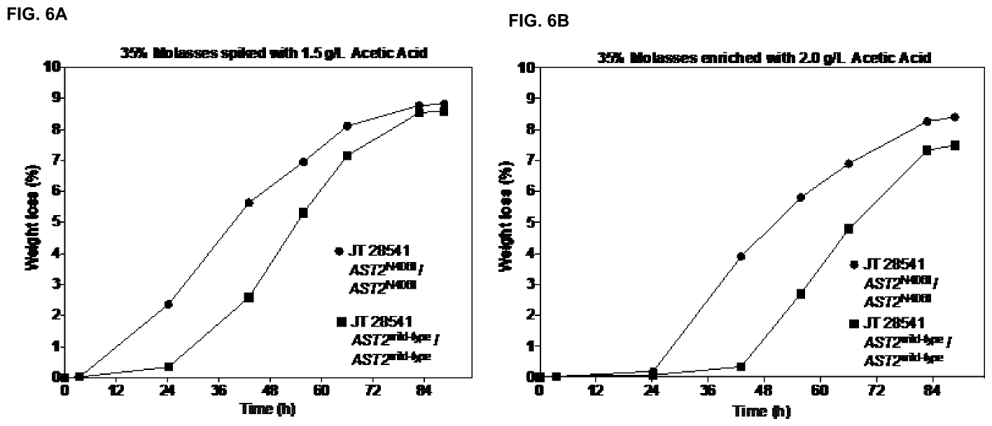

Additionally, the impact of the mutant AST2^{N406I} allele on tolerance to acetic acid was analysed. The AST2^{N406I} SNP was introduced in both alleles of JT 28541, resulting in strain JT 29040 (i.e. JT 28541 AST2^{N406I}/AST2^{N406I}). The JT 28541 strain was applied in high gravity fermentations in 35% sugarcane molasses containing 21.2% (w/v) sucrose and 2.5 g/l acetic acid. To investigate tolerance to higher acetic acid levels, this medium was additionally spiked with 1.5 g/l or 2.0 g/l of acetic acid, resulting in total acetic acid concentrations of 4.0 g/l and 4.5 g/l, respectively. Under these conditions, JT 29040 displayed an improved fermentation capacity and apparent reduction of residual sucrose levels (FIG. 6A-B).

### Example 6. Effect of AST2^{N406I} Overexpression on Tolerance to Multiple Inhibitors

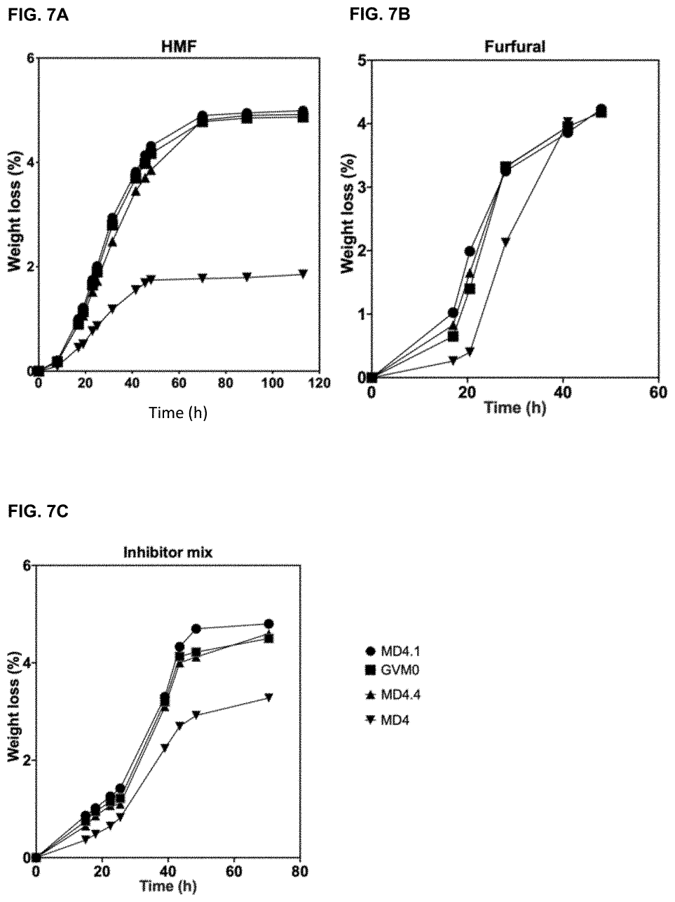

Strain MD4, which is tetraploid for AST2, was engineered to comprise one copy (MD4.1) or four copies (MD4.4) of the mutant AST2^{N406I} allele. The strains were evaluated for inhibitor tolerance in YPDX medium enriched with 12.0 g/l HMF (FIG. 7A) and in YPDX enriched with a mixture of inhibitors (2.80 g/l HMF, 1.75 g/l furfural, 0.35 g/l vanillin and 4.20 g/l acetic acid) (FIG. 7B). GVM0, the original WG transformant of MD4, with also one copy of AST2^{N406I} and three AST2 alleles was included. The results show that all strains with at least one AST2^{N406I} allele display the same degree of improvement in fermentation performance compared to the MD4 strain.

### Example 7. Effect of AST2^{N406I} Expression in Other Industrial Strain Genetic Backgrounds for Tolerance to Multiple Inhibitors

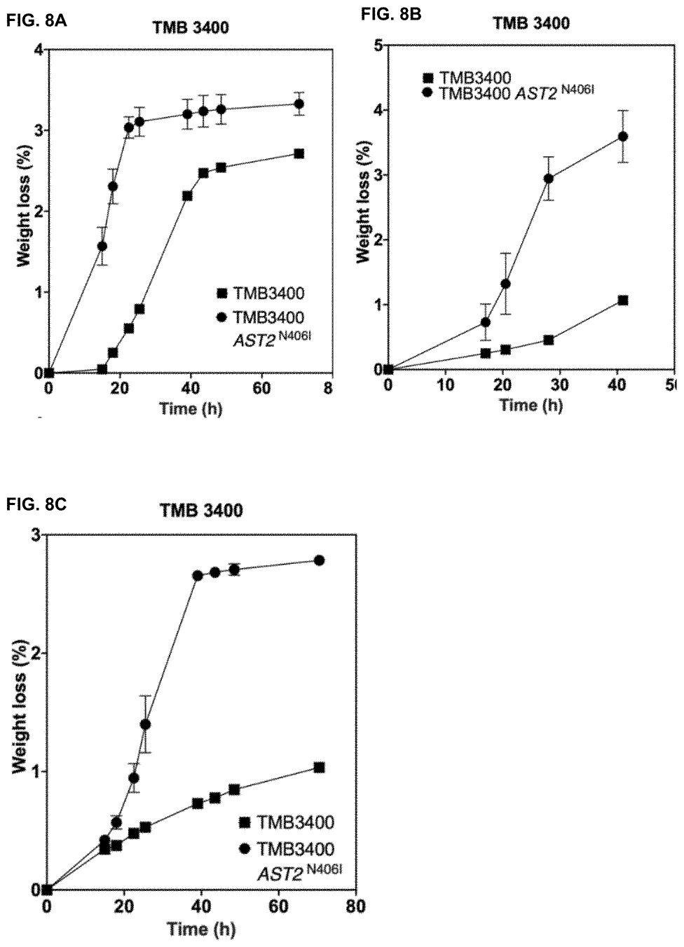

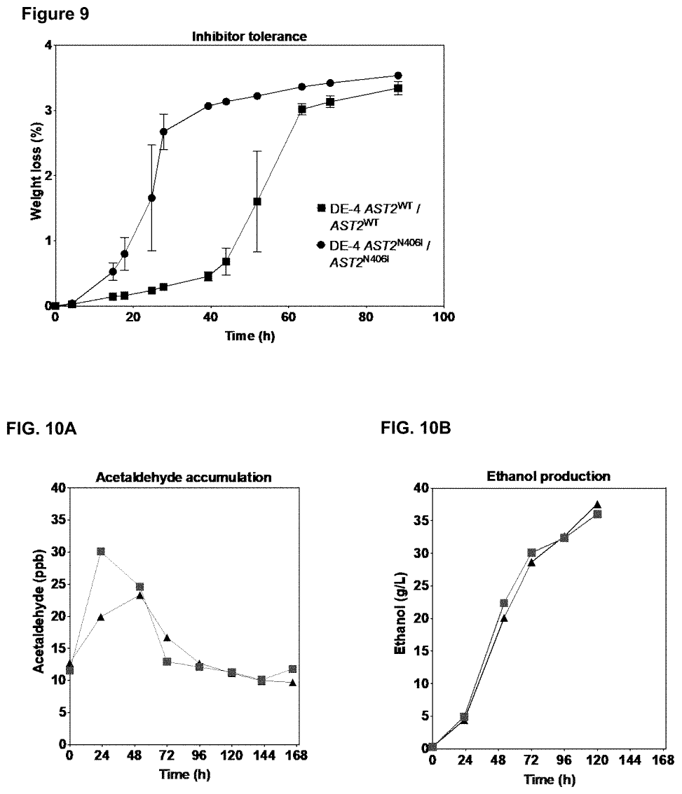

To evaluate the commercial relevance of AST2^{N406I} in improving inhibitor tolerance in yeast, 2G industrial yeast strains with different genetic backgrounds were engineered to comprise the AST2^{N406I} SNP. Insertion of AST2^{N406I} improved the fermentation capacity of TMB 3400 significantly in YPDX with 12.0 g/l HMF but also in YPDX with 4.0 g/l furfural and in YPDX enriched with a mixture of inhibitors (FIG. 8 A, B, C). The 2G industrial yeast strain DE-4, comprising approximately 16 copies of a Eubacterium spp. xylose isomerase cassette, was engineered to comprise two AST2^{N406I} copies. This resulted in strain JT 29042 (i.e. DE-4 AST2^{N406I}/AST2^{N406I}). Fermentation performance was evaluated in YPDX with 2.80 g/l HM F, 1.75 g/l furfural, 0.35 g/l vanillin and 4.20 g/l acetic acid. This revealed improved fermentation rate and yield of JT 29042 compared to DE-4 in presence of a high concentration of inhibitor mixture (FIG. 9).

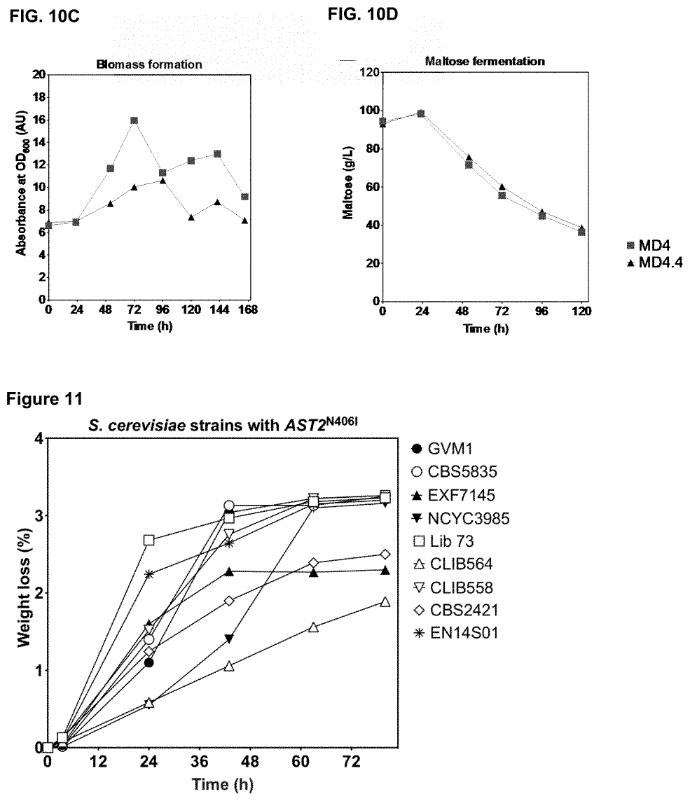

Next, the effect of the AST2^{N406I} allele was also evaluated in industrially representative settings. First, the AST2^{N406I} SNP was introduced in the industrial yeast Ethanol Red, resulting in strain JT 29034 (i.e. Ethanol Red AST2^{N406I}/AST2^{N406I}). Ethanol Red and JT 29034 were subsequently evaluated for fermentation capacity in corn mash hydrolysate. The yeast strains were propagated for 8 h in 100 g 60% corn mash 40% water, at 30° C., 250 rpm. Subsequently, the strains were evaluated in small-scale fermentations in 100 g 100% corn mash. HPLC analysis revealed 0.98% residual glucose with Ethanol Red, but only 0.90 with JT 29034. Glycerol levels ranged from 0.79% for Ethanol Red to 0.84% for JT 29034, while the ethanol titer (% w/v) observed was 18.43% for Ethanol Red but 19.22% for JT 29034. Second, fermentation capacity of MD4 and MD4.4 (i.e. MD4 with 4 copies of AST2^{N406I}) was evaluated in static fermentations in 250 ml wort. For this purpose, the yeast strains were first precultured in 3 ml YPD at 30° C. for 24 h, and subsequently in 50 ml wort at 18° C. for 72 h, prior to evaluating fermentation capacity at 14° C. GC analysis revealed that acetaldehyde accumulation was reduced in MD4.4 compared to MD4, which indicates a more rapid conversion of acetaldehyde into ethanol. Moreover, we observed that ethanol production was slightly higher at the end of the fermentation (120 h) for MD4.4 compared to MD4. In addition, biomass formation was reduced for MD4.4 compared to MD4 (FIGS. 10A-10D). This is a surprising finding and provides a solution to the inability in first generation bioethanol production to enhance ethanol yields by reducing yeast biomass formation without causing unwanted side-effects. Residual maltose was similar for MD4.4 compared to MD4, indicating that the increased ethanol titer by MD4.4 after 120 h could not be explained by improved residual maltose fermentation. No differences were observed for glucose and maltotriose fermentation, nor for glycerol formation between both yeast strains. These data illustrate that AST2^{N406I} expression increases the fermentation performance of commercial yeasts in industrially representative settings, i.e. less residual carbon source, faster conversions and increased levels of fermentation products.

### Example 8. Alternative AST2^{N406I} Alleles

We have screened the sequenced genomes of 1011 S. cerevisiae strains (Peter et al. 2018 Nature 556: 339-344) for the possible occurrence of the AST2^{N406I} SNP. Although the AST2^{N406I} SNP was present in the genome of eight strains: CBS5835, EXF7145, NCYC3985, Lib 73, CLIB564, CLIB558, CBS2421 and EN14S01, none of them comprises an AST2 allele identical to SEQ ID No. 2. Compared to GVM1, five strains had the same 4 mutations, and two other strains contained the same 5 mutations (Table 1).

TABLE 1
Homology of yeast strains with AST2^{N406I} SNP compared to GVM1.

| | Identity to | |
|---|---|---|
| | SEQ ID No. 2 | Amino Acid mutations |
| | (AST2 of strain | compared to |
| Strain Name | GVM1) in % | AST2 of strain GVM1 |
| GVM1 | 100.00 | — |
| JT 23622 | 99.69 | A3E, F185L, P346S, Y413Y |
| (EXF7145, BFN) | | |
| JT 27851 | 99.69 | A3E, F185L, P346S, Y413Y |
| (NCYC3985, ALD) | | |
| BGR (CLIB564) | 99.69 | A3E, F185L, P346S, Y413Y |
| BGK (CLIB558) | 99.69 | A3E, F185L, P346S, Y413Y |
| ARS(CBS2421) | 99.69 | A3E, F185L, P346S, Y413Y |
| JT 25468 | 99.61 | A3E, F185L, D274G, T286A, |
| (CBS5835, AHP) | | Y413Y |
| CHF (Lib 73) | 99.61 | A3E, F185L, D274G, T286A, |
| | | Y413Y |

The fermentation capacity of those 8 yeast strains was evaluated in YPDX in the presence of 12 g/l HMF. Interestingly, all these strains showed a similar fermentation capacity under these conditions compared to GVM1 (FIG. 11). This confirms that the 406I SNP is indeed causative to the effects disclosed in current application and furthermore shows that the invention is not restricted to the expression of a nucleic acid molecule encoding SEQ ID No. 2. Indeed, expression of nucleic acid molecules that encode closely similar AST2 alleles that are not identical to SEQ ID No. 2 but do comprise the 406I SNP, leads to the same increased tolerance towards fermentation inhibitors as disclosed herein.

### Example 9. Evaluation of the Effect on Inhibitor Tolerance of the Corresponding AST1^{D405I} Mutation

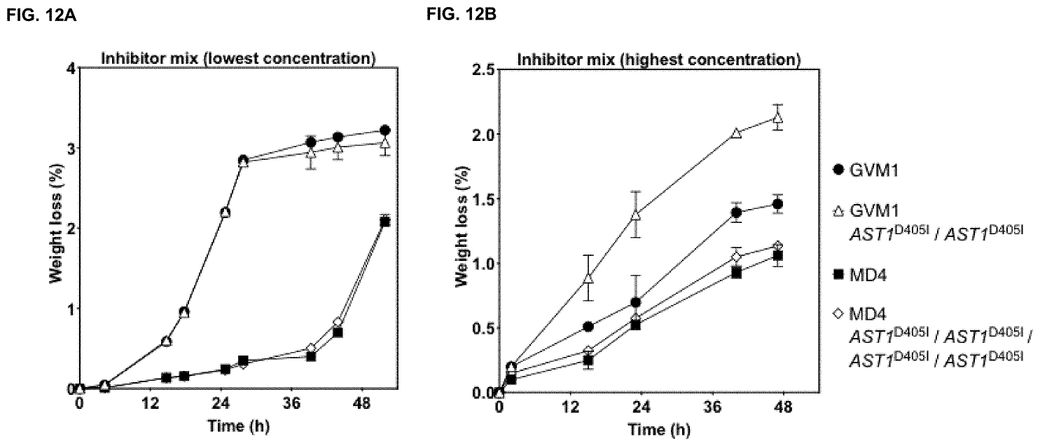

AST1 is a paralog of AST2, also belonging to the quinone oxidoreductase subfamily of the medium-chain dehydrogenase/reductase family. Ast1 and Ast2 have many conserved regions, including the domain downstream from N406 in Ast2 and the corresponding D405 in Ast1. Interestingly, the AST1^{D405I} SNP could not be found in the genomes of 1011 S. cerevisiae strains sequenced by Peter et al. (2018 Nature 556: 339-344). Two copies of the corresponding AST1^{D405I} mutation have been engineered into strain GVM1, that comprises one copy of AST2^{N406I}, and four AST1^{D405I} copies were inserted in MD4, that comprises only wild type AST2. The resulting strains were evaluated for inhibitor tolerance in fermentations in YPDX medium enriched with a mixture of inhibitors (HMF, furfural, vanillin and acetic acid) in low and high concentrations. At low inhibitor levels, the AST1^{D405I} mutation did not appear to confer any additional protective effect in the MD4 strain (only comprising wild type AST2) or in the GVM1 strain (presence of AST2^{N406I}) (FIG. 12A). However, at high inhibitor concentrations, the GVM1 strain expressing two AST1^{D405I} copies showed a better fermentation performance compared to the parent GVM1 strain (FIG. 12B). There was no significant difference in fermentation performance between the MD4 strain and the MD4 version comprising four AST1^{D405I} copies under these conditions (FIG. 12B). These results indicate that the AST1^{D405I} mutation can further enhance the protective effect of AST2^{N406I} against high concentrations of inhibitors, but that by itself it does not have a detectable effect on inhibitor tolerance under our experimental conditions.

### Experimental Procedures

Small-Scale Fermentations in Synthetic Medium and in Lignocellulose Hydrolysates

Five different lignocellulose hydrolysates were used: two bagasse hydrolysates, two corn cob hydrolysates and one spruce hydrolysate (see Table 2 for their composition).

After preculture of the yeast strains for 48 h at 30° C. with shaking at 200 rpm in YPD2% (10 g/l yeast extract, 20 g/l bacteriological peptone, 2% D-glucose) up to stationary phase, small-scale (10 ml) semi-anaerobic fermentations with MD4 and T18 were performed at pH 5.2, 35° C., shaking at 350 rpm, and a yeast inoculum OD 5.0. Weight loss of the fermentation tubes, which is correlated with CO₂ production during conversion of glucose and xylose into ethanol, was measured continuously, or sampling at different timepoints was performed to analyse sugar and inhibitor concentrations by HPLC. All other fermentations (unless specified otherwise) were also performed at pH 5.2, 35° C., shaking at 350 rpm, and a yeast inoculum OD 5.0, either in 1) YPD6.5% with 8 g/l HMF for screening of fermentation capacity of the most HMF tolerant strains from the yeast strain collection, in 2) YPD6.5%×4.0% (4% D-xylose) with 6 g/l or 12 g/l HMF, or corn cob hydrolysate 2 (see Table 2) enriched with 0.0 g/l, 0.6 g/l, 1.0 g/l or 3.0 g/l HMF to evaluate HMF tolerance in fermentations of the WG transformants of MD4 and the segregants of GVM0, in 3) YPD6.5%×4.0% with 12 g/L HMF to evaluate the genetic modification in GVM1 causative for enhanced HMF tolerance, and in 4) YPD6.5%×4.0% with 12 g/l HMF, or corn cob hydrolysate 2 enriched with 0.0 g/l, 0.6 g/l, 1.0 g/l or 3.0 g/l HMF to evaluate the effect of AST2^{N406I} in MD4 for tolerance of yeast fermentation capacity to different inhibitors and stress factors.

TABLE 2
Sugar and inhibitor composition of hydrolysates used in this study
(1, Bagasse 1; 2, Corn cob 1; 3, Bagasse 2; 4, Corn cob 2; 5, Spruce
1), and concentrations of inhibitors spiked in these hydrolysates.

| | | | | | | | Inhibitor |
|---|---|---|---|---|---|---|---|
| | | | | | | Hydrolysates | concentrations |
| Hydrolysate | | | | | | used in | added to each |
| Component | 1 | 2 | 3 | 4 | 5 | literature | hydrolysate |
| Glucose | 6.12% | 6.88% | 6.20% | 6.50% | 4.2% | | |
| Xylose | 3.92% | 5.66% | 3.66% | 4.00% | 1.3% | | |
| Arabinose | 0.20% | 0.47% | 0.27% | 0.40% | 0.32% | | |
| Acetic Acid | 0.53% | 0.63% | 0.43% | 0.60% | 0.70% | 0.10-1.30% | 0.03%, 0.06%, |
| | | | | | | | 0.10%, 0.30%, |
| | | | | | | | 0.60%, 0.80% |
| Levulinic | 0.01% | 0.00% | 0.00% | 0.00% | 0.17% | 0.01-0.32% | 0.03%, 0.06%, |
| Acid | | | | | | | 0.10%, 0.30%, |
| | | | | | | | 0.60% |
| Formic Acid | 0.06% | 0.03% | 0.03% | 0.03% | 0.03% | 0.06-0.68% | 0.03%, 0.06%, |
| | | | | | | | 0.10%, 0.30%, |
| | | | | | | | 0.60% |
| Furfural | 0.03% | 0.04% | 0.03% | 0.02% | 0.27% | 0.03-0.29% | 0.03%, 0.06%, |
| | | | | | | * | 0.10%, 0.30%, |
| | | | | | | | 0.60% |
| HMF | 0.01% | 0.01% | 0.02% | 0.02% | 0.53% | 0.01-0.59% | 0.03%, 0.06%, |
| | | | | | | | 0.10%, 0.30%, |
| | | | | | | | 0.60% |
| Vanillin | n.d. | n.d. | n.d. | n.d. | n.d. | 0.00-0.43 g/l | 0.03 g/l, 0.10 g/l, |
| | | | | | | | 0.30 g/l |
| 4-hydroxy | n.d. | n.d. | n.d. | n.d. | n.d. | 0.00-0.01 g/l | 0.001 g/l, 0.003 g/l, |
| benzoic acid | | | | | | | 0.01 g/l |
| 4-hydroxy | n.d. | n.d. | n.d. | n.d. | n.d. | 0.00-0.11 g/l | 0.003 g/l, 0.010 g/l, |
| benzaldehyde | | | | | | | 0.030 g/l |

* Note:
Concentrations of furfural present in lignocellulosic hydrolysates in literature were found to be up to 0.29%, with the exception of one reference (corn stover was found to be 1.10%, while the same lignocellulose pretreated in another fashion only contained 0.27% furfural).

Screening of Yeast Strain Collection

A yeast strain collection of 2526 S. cerevisiae strains and 17 non-conventional yeast species previously reported as displaying high tolerance to HMF during growth on solid nutrient medium (Mukherjee et al. 2017 Biotechnol Biofuels 10: 216), was screened for their level of HMF tolerance by evaluating growth after 48 h at 30° C. on solid synthetic nutrient medium (YPD2%) with 8 g/l HMF. The non-conventional yeast species screened were Candida glabrata, Metschnikowia reukaufii, Kluyveromyces marxianus (2 strains), Brettanomyces bruxellensis, Pachysolen tannophilus, Ambrosiozyma monospora, Scheffersomyces stipitis, Saccharomyces servazii (3 strains), Zygosaccharomyces bailiff (4 strains), Torulaspora delbrueckii, Issatchenkia orientalis, S. kudriazevii (2 strains), Pichia kluyverii, Debaryomyces hansenii, Meyerozyma guilliermondii, Pichia membranifaciens and Pichia anomala.

Whole-Genome Transformation and Selection of Transformants

MD4 was whole-genome (WG) transformed with gDNA from C. glabrata strain JT26560, and S. cerevisiae strains JT25869, JT23146, JT21620, JT23341, MD4, S288C, JT25416, JT25880, JT22277 and JT22689. For isolation of gDNA, yeast cells were suspended in 200 μl water and mixed with glass beads (0.45 mm) in 2 ml screw cap tubes into which 200 μl PCI solution [45.5% (v/v) phenol pH 4.2, 43.6% (v/v) chloroform, 1.8% (v/v) isoamyl alcohol, 9.1% (v/v) sodium dodecyl sulfate] was added. Cells were lysed with a FastPrep-24 Classic Instrument for 20 s at 6.0 M/s, and cell lysate was centrifuged (10 min at 14,000 rpm). 200 μl clear supernatant was mixed with 1000 μl ice-cold 99.8% ethanol, vortexed and stored at −20° C. for 1 h. The pellet was washed with 70% ethanol, resuspended in 50 μl water and sheared with the FastPrep for 60 s at 6.5 M/s to increase the fraction of smaller gDNA fragments. For WGT, 5 μg gDNA was transformed into tetraploid strain MD4 via electroporation. After 4 h recovery in 1:1 YPD2% and 1M D-sorbitol, transformants were plated on YPD6.0%×4.5% with 2.5 g/l HMF and incubated at 30° C. for 72 h. Transformants obtained were restreaked on YPD6.0%×4.5% plates with 2.5 g/l or 4.0 g/l HMF.

Transformation of S. cerevisiae

Yeast strains were transformed for introduction of plasmids for CRISPR/Cas9 targeting, to perform RHA or for whole-genome transformation. This was either achieved by electroporation according to Benatuil et al. (2010 Protein Eng Des Sel 23: 155-159) or by transformation according to Gietz and Schiestl (2007 Nat Protoc 2: 31-34).

### Sporulation of Strain GVM1

The tetraploid strain GVM0, obtained by WGT of MD4 with gDNA of C. glabrata, was sporulated to obtain diploid segregants. For that purpose, the strain was first cultured overnight in YPD2% at 30° C. and 200 rpm, subsequently inoculated into 30 ml YPD2% at OD 1 and cultivated for 6 h at 30° C. and 200 rpm until exponential phase. Cells were washed with water and plated on two solid sporulation media (1% potassium acetate, 0.25% yeast extract, 0.1% D-glucose at pH 6) and CSH (1% potassium acetate, 0.05% dextrose, 0.10% yeast extract). After lyticase treatment for 3 min at RT, single spores were isolated with a dissection microscope.

Genomic DNA Isolation, Whole-Genome Sequencing and Bio-Informatics Analysis

gDNA of strains MD4 and GVM1 was isolated with the MasterPure Yeast DNA Purification Kit (Lucigen) and submitted to whole-genome sequence analysis (Illumina) with 125 bp paired-end reads. DNA sequences were mapped by using the NGSEP pipeline (version 3.3.1) (Duitama et al. 2014 BMC Genomics 15: 207). Bowtie 2 (Langmead & Salzberg 2012 Nat Methods 9: 357-359) was used to map the genome of MD4 and GVM1 against that of S288C (version R64-2-1 at SGD). Parameters for variant calling were [-runRP -runRep -runRD -maxBaseQS 30 -minQuality 40 -maxAlnsPerStartPos 2 -knownSTRs <STR_file>]. Tandem Repeats Finder (Benson 1999 Nucleic Acids Res 27: 573-580) was used to generate an STR file of each reference genome. The combined .vcf file was filtered using parameter [-q 40] and functional annotation of genomic variants was performed with NGSEP. Further filtering was achieved with in-house scripts. In this way, a list of genomic variations between MD4 and GVM 1 was generated, which consisted of nine heterozygous non-synonymous SNPs.

### Reciprocal Hemizygosity Analysis (RHA)

RHA was performed with strain GVM1. For this purpose, a nourseothricin (clonNAT) cassette was amplified with Q5 polymerase in a medium containing 4 μl Q5 buffer, 4 μl GC enhancer, 1.6 μl dNTPs (10 mM), 1 μl forward primer (10 μM), 1 μl reverse primer (10 μM), 0.2 μl Q5 HF polymerase (New England BioLabs, NEB) and 1 ng p77 plasmid (in a 50 μl reaction volume) from plasmid pTOPO-A1-G2-B-NAT-P-G2-A2(p77) with specific primer tails for the 9 non-synonymous SNPs identified in GVM1 after WGT of MD4. PCR amplification was performed as follows: 4 min at 98° C., followed by 30 cycles consisting of 30 s at 98° C., 30 s at 70° C. and 1 min at 72° C., followed by 5 min at 72° C. The cassette generated was transformed into GVM1 by the Gietz protocol to delete each time one allele of the heterozygous gene containing a non-synonymous SNP. Transformants were subsequently plated on YPD2% with 100 μg/ml nourseothricin, and evaluated for deletion of either the wild type or the mutant allele via allele-specific PCR with TaqE polymerase [2 μl Buffer E, 2 μl dNTPs (10 mM), 1 μl forward primer (10 μM), 1 μl reverse primer (10 μM), 0.5 μl TaqE polymerase, 1 μl gDNA (100 ng/μl) in 20 μl total volume]. PCR amplification was carried out as follows: 4 min at 94° C., followed by 30 cycles of 25 s at 94° C., 25 s at 55° C., and 45 s at 72° C.), followed by 5 min at 72° C. Correct deletion of the two alleles was confirmed by Sanger sequencing (Mix2Seq at Eurofins).

CRISPR/Cas9 Genome Editing

CRISPR/Cas9 genome editing was performed to introduce multiple copies of AST2^{N406I} in strains MD4, GVM1, DE-4, Ethanol Red and TMB3400; and also to introduce AST1^{D405I} in MD4. To perform CRISPR/Cas9 in S. cerevisiae strains, guide RNAs (gRNAs) were designed based on the whole-genome sequence data of the strains to be modified. The CRISPR/Cas9 plasmids (from Streptococcus pyogenes) used were modified from (Mali et al. 2013 Science 339: 823-826) as follows. The hCas9 plasmid (Addgene #41815) was modified with a KanMX cassette in order to select transformants on solid nutrient plates with geneticin (plasmid p51-KanMX). The gRNA_Cloning Vector (Addgene #41824) was modified with a NatMX cassette in order to select transformants on solid nutrient plates with nourseothricin (plasmid p59-NAT). Based on on-target activity, aspecific cleaving (determined via a blast search of 12 bp from the 3′ end of the gRNA followed by NGG, NGA or NAG), proximity to AST2^{N406I} or AST1^{D405I}, absence of a stretch of five or more thymines, we selected the most efficient gRNA, 5′-TTATTCCTGGAAAAATTTCA-3′, to target AST2 and 5′-TATAAGAAAATGCTTCTTTA-3′ to target AST1. A linear donor fragment containing the AST2^{N406I} or AST1°^{405I} mutation was used to repair the double strand break after CRISPR/Cas9 targeting.

After restriction digestion with XhoI (NEB) and EcoRV (NEB), the gRNA was cloned in plasmid p59 using Gibson assembly (NEB), in a reaction with 50 ng plasmid and 3 times molar excess of the gRNA insert, followed by incubation at 50° C. for 1 h. Two μl of the ligation mixture was transformed into DH5alpha Escherichia coli cells that were previously made competent with RbCI treatment (Li 2011 Bio-protocol 1: e76). Cells were incubated for 30 min on ice, heat shocked for 45 s at 42° C., and incubated again for 5 min on ice. Next, 1 ml LB medium (10 g/l tryptone (Oxoid), 5 g/l yeast granulated extract (Merck), and 1 g/l NaCl 99.5% were added, and the cells were incubated at 37° C. and 300 rpm for 1 h. Subsequently, the transformed E. coli cells were plated on solid LB plates with 100 μg/ml ampicillin, and incubated overnight at 37° C. Next, plasmid p59-NAT-gRNA-AST2 was purified with NucleoSpin Plasmid EasyPure (Macherey-Nagel). Thereafter, p51KanMX and subsequently p59-NAT-gRNA-AST2 or p59-NAT-gRNA-AST1, as well as the linear AST2^{N406I} or AST1^{D405I}, were transformed into strains MD4 and GVM1 via electroporation. After loss of the two plasmids by subculturing under non-selective conditions, the transformants were analyzed by allele-specific PCR and Sanger sequencing.

HPLC

For HPLC, a Bio-Rad Aminex HPX 87H 300X7 8 mm column was used. The eluents was H₂SO₄.

SEQUENCES
SEQ ID No. 1
MAEKILENKDPKLEAMTVDHEVSAPKPIPVDEPTLTRVARPLRHVRH
IPVKSLVFHSKHGPITFSYENKIKLPISKNKLVVQVNYVGLNPVDMK
IRNGYTKPIYGEAGIGREYSGVITHVGDNLTNRWNVGDDVYGIYYHP
KLAIGALQSSLLIDPRVDPILMRPKNTLSPEKAAGSLFCLGTALNLL
AQLKEKDQLNTESNVLINGGTSSVGMFAIQLLKRYYKVSKKLVVVTS
GNGAAVLSEHFPDLKDEIIFINYLSCRGKSSKPLRRMLDTGKVVDYD
DFNTLKETEDYTQGKFNVVLDFIGGYDILSHSSSLIHAKGAYITTVG
DYVGNYKKDVFDSWDNPSANARKMFGSMLWSYDYSHFYFDPNIKIIP
KKNDWIHECGKLLNEGVVDCVVDKVYSWKNFKEAFSYMATQRAQGKL
IMKVEGF*
SEQ ID No. 2
MAEKILENKDPKLEAMTVDHEVSAPKPIPVDEPTLTRVARPLRHVRH
IPVKSLVFHSKHGPITFSYENKIKLPISKNKLVVQVNYVGLNPVDMK
IRNGYTKPIYGEAGIGREYSGVITHVGDNLTNRWNVGDDVYGIYYHP
KLAIGALQSSLLIDPRVDPILMRPKNTLSPEKAAGSLFCLGTALNLL
AQLKEKDQLNTESNVLINGGTSSVGMFAIQLLKRYYKVSKKLVVVTS
GNGAAVLSEHFPDLKDEIIFINYLSCRGKSSKPLRRMLDTGKVVDYD
DFNTLKETEDYTQGKFNVVLDFIGGYDILSHSSSLIHAKGAYITTVG
DYVGNYKKDVFDSWDNPSANARKMFGSMLWSYDYSHFYFDPNIKIIP
KKNDWIHECGKLLNEGVVDCVVDKVYSWKIFKEAFSYMATQRAQGKL
IMKVEGF*
SEQ ID No. 3
MAKDILKNQDPKLQAMIVEHSAPAPKEIPMDAPVLKRVARPLRHVKF
IPIKSLIFHTKTGPMDFSYEKKIKTPIPKNKIVVRVSNVGLNPVDMK
IRNGYTSSIYGEIGLGREYSGVITEVGENLNYAWHVGDEVYGIYYHP
HLAVGCLQSSILVDPKVDPILLRPESVSAEEAAGSLFCLATGYNILN
KLSKNKYLKQDSNVLINGGTSSVGMFVIQLLKRHYKLQKKLVIVTSA
NGPQVLQEKFPDLADEMIFIDYLTCRGKSSKPLRKMLEEKKISQYDP
VEDKETILNYNEGKFDVVLDFVGGYDILSHSSSLIHGGGAYVTTVGD
YVANYKEDIFDSWDNPSANARKMFGSIIWSYNYTHYYFDPNAKTASA
NNDWIEQCGDFLKNGTVKCVVDKVYDWKDHKEAFSYMATQRAQGKLI
MNVEKF*
SEQ ID No. 4
MAKDILKNQDPKLQAMIVEHSAPAPKEIPMDAPVLKRVARPLRHVKF
IPIKSLIFHTKTGPMDFSYEKKIKTPIPKNKIVVRVSNVGLNPVDMK
IRNGYTSSIYGEIGLGREYSGVITEVGENLNYAWHVGDEVYGIYYHP
HLAVGCLQSSILVDPKVDPILLRPESVSAEEAAGSLFCLATGYNILN
KLSKNKYLKQDSNVLINGGTSSVGMFVIQLLKRHYKLQKKLVIVTSA
NGPQVLQEKFPDLADEMIFIDYLTCRGKSSKPLRKMLEEKKISQYDP
VEDKETILNYNEGKFDVVLDFVGGYDILSHSSSLIHGGGAYVTTVGD
YVANYKEDIFDSWDNPSANARKMFGSIIWSYNYTHYYFDPNAKTASA
NNDWIEQCGDFLKNGTVKCVVDKVYDWKIHKEAFSYMATQRAQGKLI
MNVEKF*

1. A nucleic acid molecule encoding SEQ ID No. 2.

2. A chimeric gene comprising the following operatively linked elements:
a. a promoter which is active in a eukaryotic cell,
b. a nucleic acid molecule encoding an amino acid sequence with a sequence identity of at least 90% to SEQ ID No. 1, wherein the amino acid sequence comprises an isoleucine residue at a position corresponding to position 406 of SEQ ID No. 1, and
c. a 3′ end region involved in transcription termination and/or polyadenylation.

3. The chimeric gene of claim 2, wherein the amino acid sequence with a sequence identity of at least 90% to SEQ ID No. 1 is SEQ ID No. 2.

4. The chimeric gene of claim 2, wherein the chimeric gene is comprised in a vector.

5. The chimeric gene of claim 4, wherein the vector is comprised in a yeast.

6. The chimeric gene of claim 5, wherein the yeast is a xylose fermenting yeast.

7. The chimeric gene of claim 4, wherein the yeast is able to metabolize lignocellulosic hydrolysates comprising one or more growth inhibiting compounds selected from the group consisting of hydroxymethylfurfural, furfural, formic acid, acetic acid, levulinic acid, 4-hydroxybenzoic acid, 4-hydroxybenzaldehyde, and vanillin.

8. The chimeric gene of claim 4, wherein the yeast further comprises a nucleic acid molecule encoding an amino acid sequence with sequence identity of at least 90% to SEQ ID No. 3, wherein the amino acid sequence comprises an isoleucine residue at a position corresponding to position 405 of SEQ ID No. 3.

9. The chimeric gene of claim 4, wherein the yeast is comprised in a biologically pure culture.

10. The chimeric gene of claim 4, wherein the yeast is comprised in a culture comprising lignocellulosic hydrolysates.

11. A method of providing tolerance to a fermentation inhibitor in yeast or to reduce the production of acetaldehyde in yeast fermentation, the method comprising:
introducing into the yeast a nucleic acid molecule encoding an amino acid sequence with a sequence identity of at least 90% to SEQ ID No. 1, wherein the amino acid sequence comprises an isoleucine residue at a position corresponding to position 406 of SEQ ID No. 1;
wherein the inhibitor is selected from the group consisting of hydroxymethylfurfural, furfural, formic acid, acetic acid, levulinic acid, 4-hydroxybenzoic acid, 4-hydroxybenzaldehyde, and vanillin.

12. (canceled)

13. A method of producing a fermentation product, the method comprising:
a. yeast fermenting a carbon source into a fermentation product in a medium comprising a yeast, the carbon source, and one or more growth inhibiting compounds selected from the group consisting of hydroxymethylfurfural, furfural, formic acid, acetic acid, levulinic acid, 4-hydroxybenzoic acid, 4-hydroxybenzaldehyde and vanillin; and optionally
b. recovering the fermentation product;
wherein the yeast comprises a nucleic acid molecule encoding an amino acid sequence with a sequence identity of at least 90% to SEQ ID No. 1 operatively linked to a promoter which is active in a eukaryotic cell and a 3′ end region involved in transcription termination and/or polyadenylation.

14. The method according to claim 13, where the fermentation product is ethanol, isobutanol, lactic acid, 2,3-butanediol, muconic acid, protocatechuic acid, 3-hydroxy-propionic acid, acrylic acid, acetic acid, succinic acid, citric acid, amino acids, 1,3-propane-diol, ethylene, glycerol, butyric acid, caproate, butanol, glyoxylate, fatty alcohols, fatty acids, β-lactam antibiotics, or cephalosporins.

15. (canceled)

16. The method according to claim 11 wherein the nucleic acid encodes an Ast2 protein comprising an N406I mutation.
